(19) United States
(12) Patent Application Publication
(10) Pub. No.: US 2026/0146018 A1
(43) Pub. Date: May 28, 2026

(54) INTERMOLECULAR BETA-METHYLENE C-H ARYLATION OF FREE ALIPHATIC ACIDS

(71) Applicant: THE SCRIPPS RESEARCH INSTITUTE, La Jolla, CA (US)

(72) Inventors: Jin-Quan YU, San Diego, CA (US); Liang HU, San Diego, CA (US); Guangrong MENG, San Diego, CA (US)

(21) Appl. No.: 19/124,070

(22) PCT Filed: Oct. 13, 2023
(86) PCT No.: PCT/US2023/076851

§ 371 Date: Apr. 24, 2025

Related U.S. Application Data
(60) Provisional application No. 63/420,048, filed on Oct. 27, 2022.

(51) Int. Cl. C07C 51/353; B01J 31/18

(52) U.S. Cl. CPC C07C 51/353; B01J 31/184; B01J 2231/46; B01J 2531/824

(57) ABSTRACT

The application provides methods of β-methylene C (sp³)-H arylation of diverse free aliphatic acids using bidentate pyridine-pyridone ligands. Herein disclosed are the first examples of methods of Pd(II)-catalyzed intermolecular β-methylene C (sp³)-H arylation of aliphatic carboxylic acids.

### CROSS REFERENCE TO RELATED APPLICATION

This application claims priority to U.S. provisional patent application No. 63/420,048, which was filed on Oct. 27, 2022, and which is hereby incorporated by reference in its entirety.

### GOVERNMENT SUPPORT

This invention was made with government support under GM084019 awarded by the National Institutes of Health. The government has certain rights in the invention.

### FIELD OF THE INVENTION

The application relates to methods of intermolecular β-methylene C(sp³)-H arylation of diverse free aliphatic acids using bidentate pyridine-pyridone ligands. Herein disclosed are the first examples of methods of Pd(II)-catalyzed intermolecular β-methylene C(sp³)-H arylation of aliphatic carboxylic acids.

### BACKGROUND OF THE INVENTION

In recent years, transition-metal-catalyzed C—H functionalization has emerged as an efficient strategy for the construction of carbon-carbon and carbon-heteroatom bonds.¹ In particular, the β-C(sp³)-H functionalization of carboxylic acid derivatives using carefully designed exogenous directing groups (DGs) has been extensively developed as potential alternative disconnections for conjugate addition.²⁻³ Notably, methylene C—H functionalization often requires the installation of bidentate directing groups as exemplified by the Daugulis amino-quinoline DG.⁴ In order to develop ligand-accelerated C—H activation reactions, a ligand-enabled strategy for the arylation of β-methylene C(sp³)-H bonds using simple monodentate amide auxiliaries was subsequently reported.⁵ However, despite the impressive disconnections they enable, the synthetic efficiency of these methodologies are limited by the need to install and remove the DGs they require (Scheme 1A). Thus, the achievement of direct C—H arylation of free carboxylic acids was deemed highly desirable due to superior atom and step economies.

Over the past decade, research has focused on ligand-enabled C—H activation reactions directed by native functional groups such as free carboxylic acids, free aliphatic amines, and

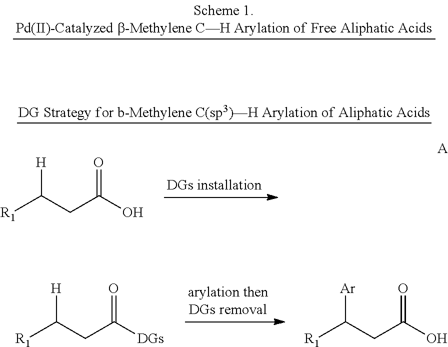

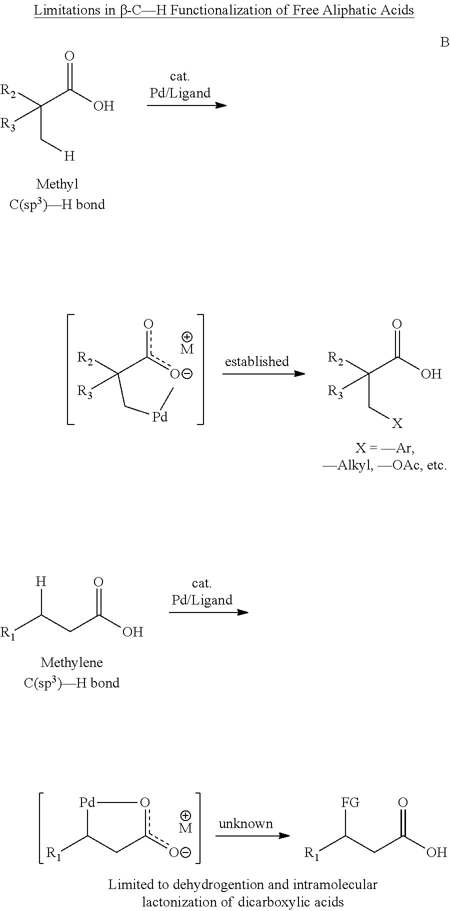

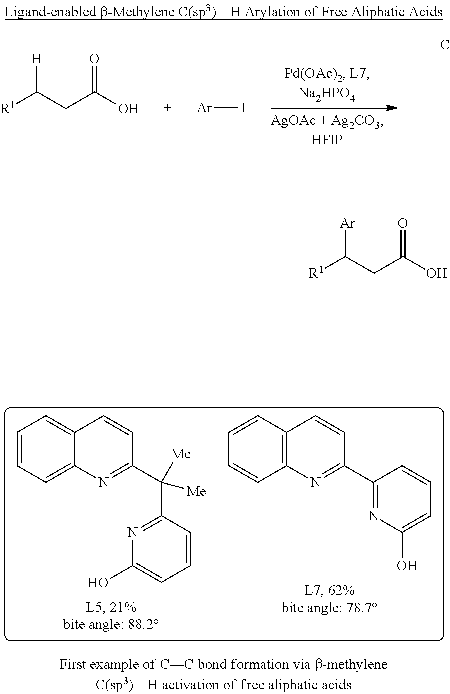

native amides.⁶ Catalytic β-C—H activation of methyl C—H bonds has led to the development of a diverse range of reactions such as arylation,^{7a-7f} olefination,^{7g} acetoxylation,^{7h,7j} and lactonization⁷ⁱ (Scheme 1B).⁷ However, methylene C—H arylation of free acid remained to be demonstrated despite the early report of methyl C—H arylation of free acid.^{7a-7f} Recently, a pair of Pd(II)-catalyzed α,β-dehydrogenation reactions of free carboxylic acids through β-methylene C—H activation, delivering α,β-unsaturated carboxylic acids or γ-alkylidene butenolides were developed.⁸ Later, in 2022, site-selective β- and γ-methylene C—H lactonization of dicarboxylic acids (Scheme 1B) was achieved.⁹ However, this chemistry was limited to intramolecular functionalization of the methylene C(sp³)-H bond.

Thus, the extrapolation of this chemistry into intermolecular reactions was expected to offer the potential to rapidly expand molecular diversity in the functionalization of β-methylene C(sp³)-H bonds. Accordingly, the expansion of this chemistry to enable intermolecular functionalization of β-methylene C(sp³)-H bonds was considered to be a particularly important goal.

Therefore, there exists a need in the field of synthetic organic chemistry, including medicinal chemistry applications, for methods of intermolecular functionalization of β-methylene C(sp³)-H bonds.

### SUMMARY OF THE INVENTION

Herein, disclosed is the first method of Pd(II)-catalyzed intermolecular β-methylene C(sp³)-H arylation of free aliphatic acids (Scheme 1C). A wide range of aliphatic carboxylic acids can thereby be directly arylated through activation of methylene C—H bonds as enabled by the herein disclosed bidentate pyridine-pyridone ligands.

The application provides a method of β-methylene C(sp³)-H arylation of aliphatic carboxylic acids, comprising treating an aliphatic carboxylic acid with an aryl iodide in the presence of a bidentate pyridine-pyridone ligand (L) and a Pd(II) catalyst.

The application provides the above method, wherein the method of β-methylene C(sp³)-H arylation of aliphatic carboxylic acids occurs according to the following reaction scheme:

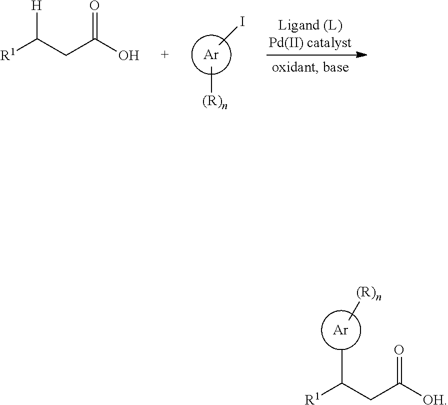

wherein:
Ar is (C₆-C₁₀)aryl;
R is H, halo, OH, (C₁-C₆)alkyl, halo (C₁-C₆)alkyl, (C₁-C₆)heteroalkyl, halo (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, Ac, —OAc, —NO₂, Ts, —OTBS, —C(═O)H, NH₂, —C(═O)(C₁-C₆)alkyl, —C(═O)O(C₁-C₆)alkyl, oxo, —S(═O)(C₁-C₆)alkyl, —S(═O)₂(C₁-C₆)alkyl, or —C(═O)NH(C₁-C₆)alkyl;
R¹ is H or selected from the group consisting of (C₁-C₁₂)alkyl, (C₂₋₁₂)alkenyl, (C₂₋₁₂)alkynyl, (C₁-C₁₀)heteroalkyl, hetero (C₂₋₁₀)alkenyl, hetero (C₂₋₁₀)alkynyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, each independently and optionally substituted with one or more R¹′;
   each R¹′ is independently OH, halo, CN, (C₁-C₆)alkyl, halo (C₁-C₆)alkyl, (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, NH₂, —C(═O)(C₁-C₆)alkyl, —C(═O)O(C₁-C₆)alkyl, oxo, Ts, —S(═O)₂F, —OS(═O)₂F, or —S(═O)₂(C₁-C₆)alkyl; and
n is 0, 1, 2, or 3.

The application provides the above method, wherein the method of β-methylene C(sp³)-H arylation of aliphatic carboxylic acids occurs according to the following reaction scheme:

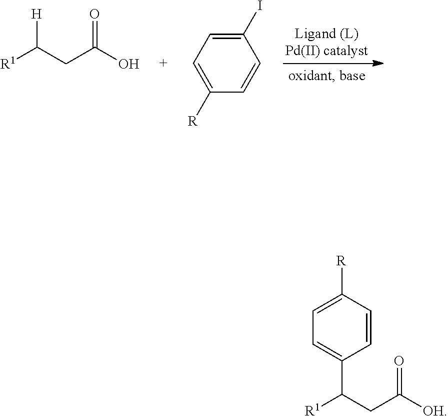

The application provides the above methods, wherein L is selected from the group consisting of:

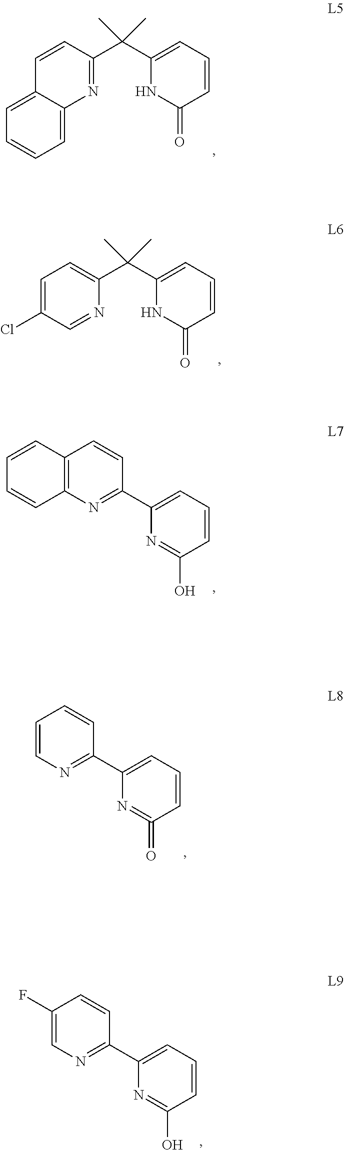

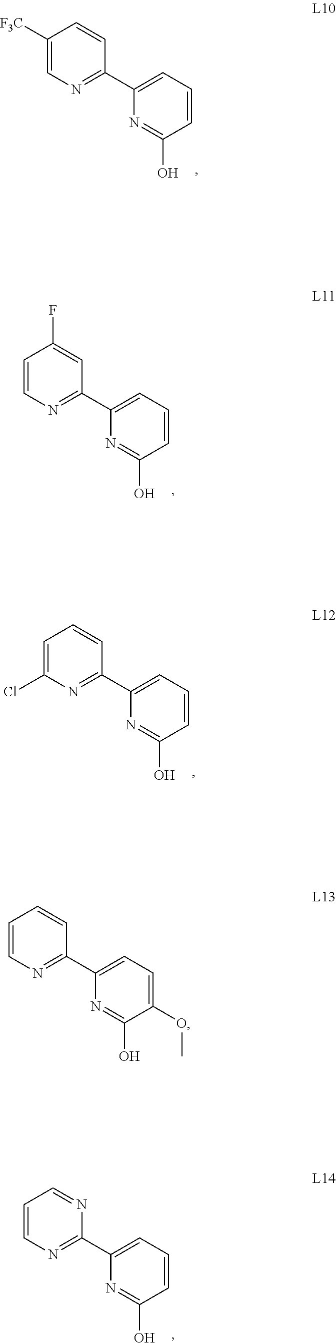 [SMILES: Oc1cccc(-c2ccc(C(F)(F)F)cn2)n1 ; Oc1cccc(-c2cc(F)ccn2)n1 ; Oc1cccc(-c2cccc(Cl)n2)n1 ; COc1ccc(-c2ccccn2)nc1O ; Oc1cccc(-c2ncccn2)n1]

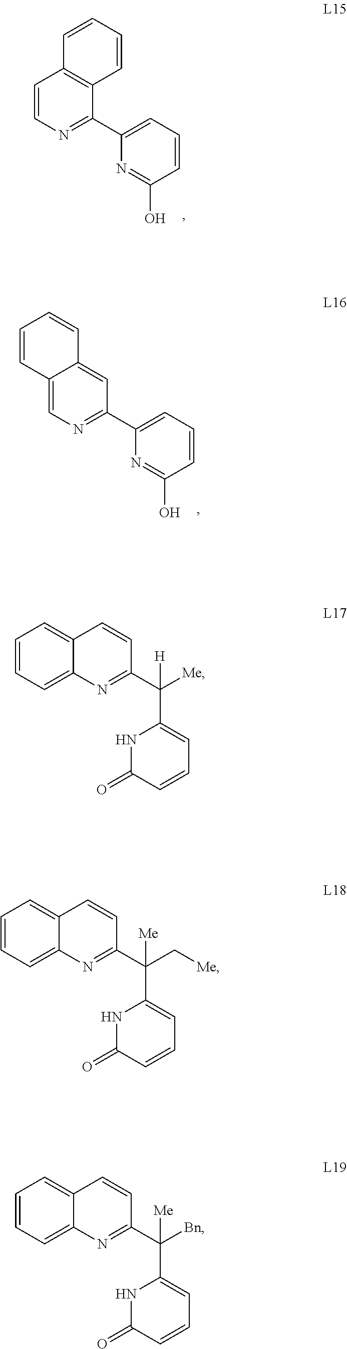 [SMILES: Oc1cccc(-c2nccc3ccccc23)n1 ; Oc1cccc(-c2cc3ccccc3cn2)n1 ; CC(c1ccc2ccccc2n1)c1cccc(=O)[nH]1 ; CCC(C)(c1ccc2ccccc2n1)c1cccc(=O)[nH]1 ; CC(Cc1ccccc1)(c1ccc2ccccc2n1)c1cccc(=O)[nH]1]

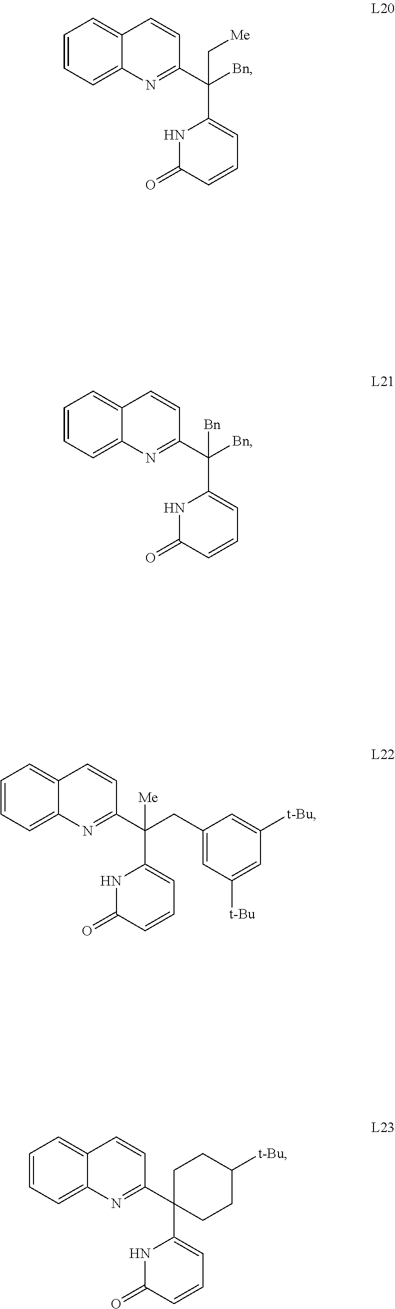 [SMILES: CCC(Cc1ccccc1)(c1ccc2ccccc2n1)c1cccc(=O)[nH]1 ; O=c1cccc(C(Cc2ccccc2)(Cc2ccccc2)c2ccc3ccccc3n2)[nH]1 ; CC(C)(C)c1cc(CC(C)(c2ccc3ccccc3n2)c2cccc(=O)[nH]2)cc(C(C)(C)C)c1 ; CC(C)(C)C1CCC(c2ccc3ccccc3n2)(c2cccc(=O)[nH]2)CC1]

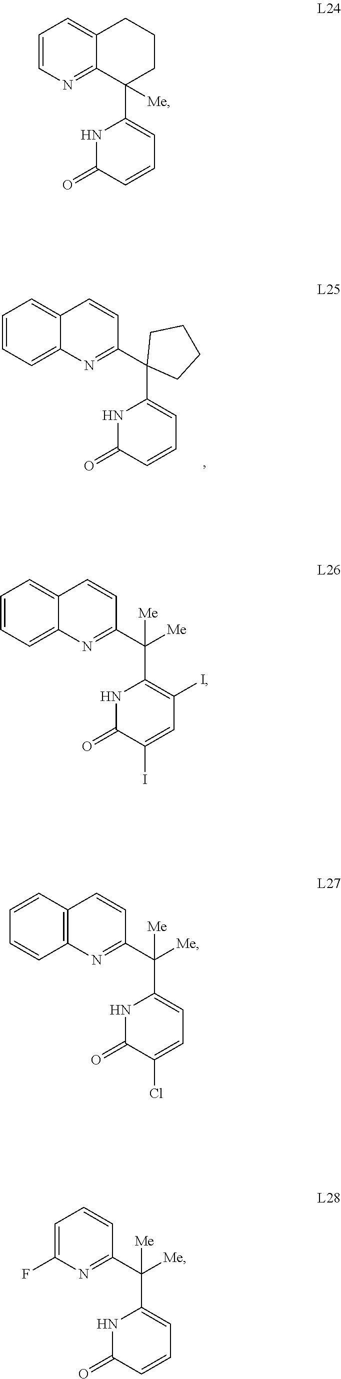 [SMILES: CC1(c2cccc(=O)[nH]2)CCCc2cccnc21 ; O=c1cccc(C2(c3ccc4ccccc4n3)CCCC2)[nH]1 ; CC(C)(c1ccc2ccccc2n1)c1[nH]c(=O)c(I)cc1I ; CC(C)(c1ccc2ccccc2n1)c1ccc(Cl)c(=O)[nH]1 ; CC(C)(c1cccc(F)n1)c1cccc(=O)[nH]1]

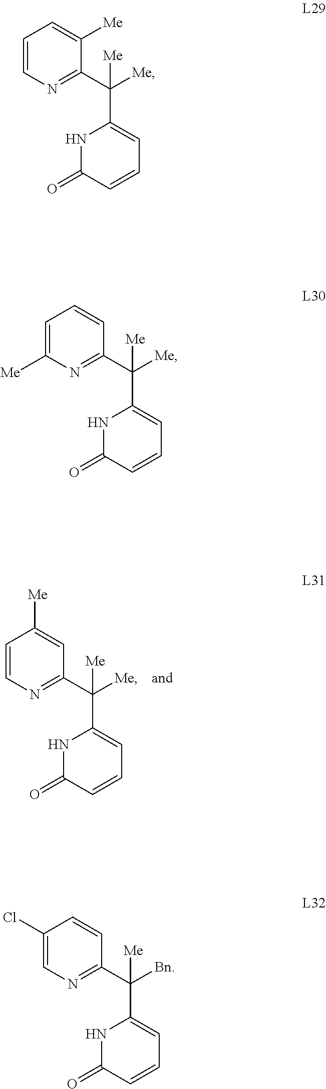 [SMILES: Cc1cccnc1C(C)(C)c1cccc(=O)[nH]1 ; Cc1cccc(C(C)(C)c2cccc(=O)[nH]2)n1 ; Cc1ccnc(C(C)(C)c2cccc(=O)[nH]2)c1 ; CC(Cc1ccccc1)(c1ccc(Cl)cn1)c1cccc(=O)[nH]1]

The application provides the above methods of β-methylene C(sp³)-H arylation of aliphatic carboxylic acids, wherein the bidentate pyridine-pyridone ligand L is L7.

The application provides the above methods, wherein the method of β-methylene C(sp³)-H arylation of aliphatic carboxylic acids occurs according to the following reaction scheme:

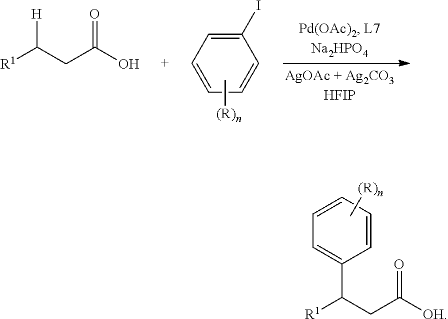

### DETAILED DESCRIPTION OF THE INVENTION

In the quest for methods of intermolecular functionalization of β-methylene C(sp³)-H bonds, efforts began by testing a range of ligands for the Pd(OAc)₂-catalyzed reaction of model substrate butyric acid (1a, 1.0 equiv.) with aryl iodide (4-iodotoluene, 2.0 equiv.) (Table 1). No desired product was obtained in the absence of ligand. Similarly, a representative monodentate pyridine-type ligand (L2), a mono-dentate pyridone ligand (L3) and an N-acetyl amino acid (MPAA) ligand (L4), also failed to deliver any product. Attention was then turned toward pyridone-based bidentate ligands. Recent findings have demonstrated that these types of bidentate ligands can promote carboxylic acid directed β-methylene C—H cleavage.⁸ In fact, the β-methylene arylated product was formed in a modest 21% yield when pyridine-pyridone ligand L5, which forms a six membered bidentate chelate, was employed. Encouraged by this result, next screened was a range of other six-member pyridone-based bidentate ligands, but all 6-membered chelates tested gave low yields, as described hereinbelow. The next aim was to accelerate C—H cleavage through changing the ligand bite angle (L7) based on the structure of LP. Indeed, the five-member quinoline-pyridone bidentate ligand improved the yield to 62% (L7). Encouraged by this result, the electronic and/or steric properties of L7 were accordingly modified. However, efforts to modify the substitution on the ligand backbone did not further enhance the reactivity (L8-L14). In addition, a ligand variant bearing an isoquinoline moiety resulted in only 22% yield (L15).

TABLE 1
Investigation of Ligands for the β-Methylene C(sp³)-H Arylation of Free Aliphatic
Acids^{a,b}

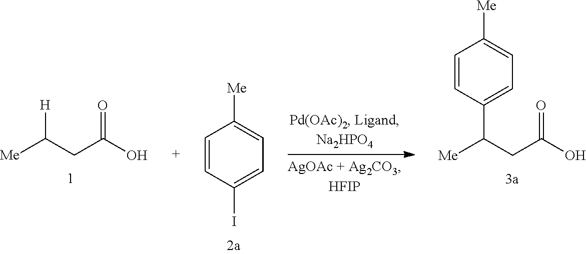 [SMILES: CCCC(=O)O ; Cc1ccc(I)cc1 ; Cc1ccc(C(C)CC(=O)O)cc1]

w/o ligand(L)
L1, 0%

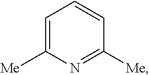

L2, 0%

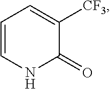 [SMILES: O=c1[nH]cccc1C(F)(F)F]

L3, 0%

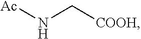

L4, 0%

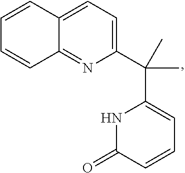 [SMILES: CC(C)(c1ccc2ccccc2n1)c1cccc(=O)[nH]1]

L5, 21%

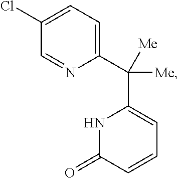 [SMILES: CC(C)(c1ccc(Cl)cn1)c1cccc(=O)[nH]1]

L6, 14%

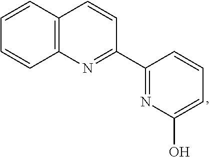 [SMILES: Oc1cccc(-c2ccc3ccccc3n2)n1]

L7, 62%

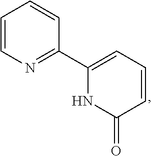 [SMILES: O=c1cccc(-c2ccccn2)[nH]1]

L8, 27%

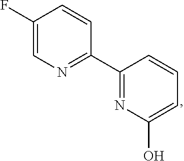 [SMILES: Oc1cccc(-c2ccc(F)cn2)n1]

L9, 16%

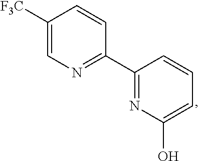 [SMILES: Oc1cccc(-c2ccc(C(F)(F)F)cn2)n1]

L10, 18%

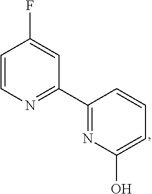 [SMILES: Oc1cccc(-c2cc(F)ccn2)n1]

L11, 20%

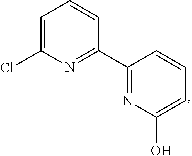 [SMILES: Oc1cccc(-c2cccc(Cl)n2)n1]

L12, 30%

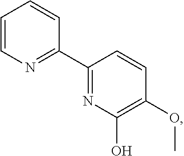

L13, 14%

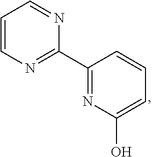 [SMILES: Oc1cccc(-c2ncccn2)n1]

L14, 28%

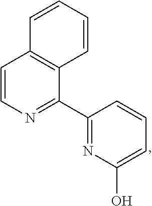 [SMILES: Oc1cccc(-c2nccc3ccccc23)n1]

L15, 22%

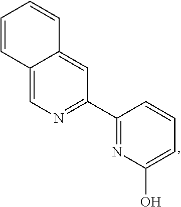 [SMILES: Oc1cccc(-c2cc3ccccc3cn2)n1]

L16, trace
^{a}Conditions: 1 (0.1 mmol), aryl iodide 2a (0.2 mmol), Pd(OAc)₂ (10 mol %), ligand (12 mol %), Na₂HPO₄ (1.0 equiv.), AgOAc (2.0 equiv.), Ag₂CO₃ (0.5 equiv.), HFIP (1.0 mL), 100° C., 48 h.
^{b}Isolated yields.

With the optimal ligand and reaction conditions in hand, a wide range of aryl iodides were tested (Table 2). Reactions employing electron-neutral or electron-rich aryl iodides (3a-3f) proved particularly effective, while aryl iodides containing electron-withdrawing groups tended to result in slightly lower yields (3g-3l). Additionally, both meta-substituted and ortho-substituted aryl iodides reagents were viable coupling partners (3m-3r). Furthermore, the reaction worked well with 3,5-disubstituted aryl iodide, giving the arylated product in 56% yield (3s). In addition to substituted phenyl iodides, heteroaryl iodides are also tolerated by this reaction (3t-3u).

TABLE 2
Aryl Iodide Scope for the β-Methylene C(sp³)-H Arylation of Free Aliphatic
Acids^{a,b}

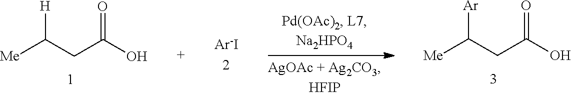

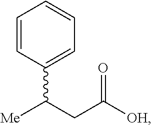 [SMILES: CC(CC(=O)O)c1ccccc1]

3b, 57%

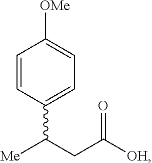 [SMILES: COc1ccc(C(C)CC(=O)O)cc1]

3c, 60%

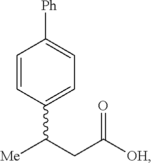 [SMILES: CC(CC(=O)O)c1ccc(-c2ccccc2)cc1]

3d, 63%

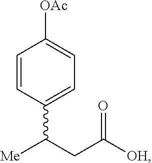 [SMILES: CC(=O)Oc1ccc(C(C)CC(=O)O)cc1]

3e, 53%

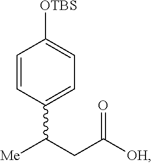 [SMILES: CC(CC(=O)O)c1ccc(O[Si](C)(C)C(C)(C)C)cc1]

3f, 55%

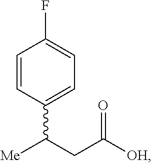 [SMILES: CC(CC(=O)O)c1ccc(F)cc1]

3g, 50%

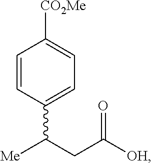 [SMILES: COC(=O)c1ccc(C(C)CC(=O)O)cc1]

3h, 53%

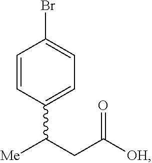 [SMILES: CC(CC(=O)O)c1ccc(Br)cc1]

3i, 44%

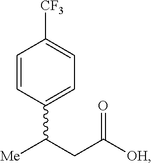 [SMILES: CC(CC(=O)O)c1ccc(C(F)(F)F)cc1]

3j, 47%

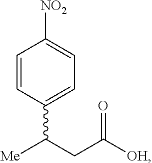 [SMILES: CC(CC(=O)O)c1ccc([N+](=O)[O-])cc1]

3k, 40%

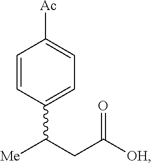 [SMILES: CC(=O)c1ccc(C(C)CC(=O)O)cc1]

3l, 51%

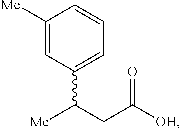 [SMILES: Cc1cccc(C(C)CC(=O)O)c1]

3m, 62%

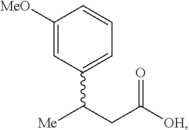 [SMILES: COc1cccc(C(C)CC(=O)O)c1]

3n, 60%

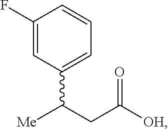 [SMILES: CC(CC(=O)O)c1cccc(F)c1]

3o, 54%

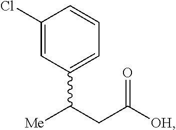 [SMILES: CC(CC(=O)O)c1cccc(Cl)c1]

3p, 55%

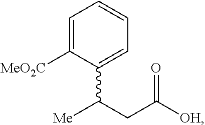 [SMILES: COC(=O)c1ccccc1C(C)CC(=O)O]

3q, 50%

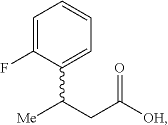 [SMILES: CC(CC(=O)O)c1ccccc1F]

3r, 53%

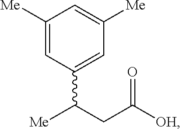 [SMILES: Cc1cc(C)cc(C(C)CC(=O)O)c1]

3s, 56%

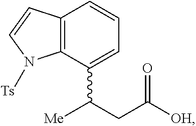 [SMILES: Cc1ccc(S(=O)(=O)n2ccc3cccc(C(C)CC(=O)O)c32)cc1]

3t, 52%

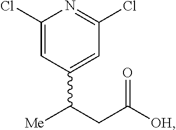 [SMILES: CC(CC(=O)O)c1cc(Cl)nc(Cl)c1]

3u, 37%
^{a}Conditions: 1 (0.1 mmol), aryl iodide 2 (0.2 mmol), Pd(OAc)₂ (10 mol %), L7 (12 mol %), Na₂HPO₄ (1.0 equiv.), AgOAc (2.0 equiv.), Ag₂CO₃ (0.5 equiv.), HFIP (1.0 mL), 100° C., 48 h.
^{b}Isolated yields.

Subsequently, arylation of a wide range of free carboxylic acids with β-methylene C—H bonds was carried out under the optimized reaction conditions (Table 3). Simple linear and

TABLE 3
Aliphatic Acid Scope for the β-Methylene C(sp³)-H Arylation of Free Aliphatic
Acids^{a,b,c,d,e}

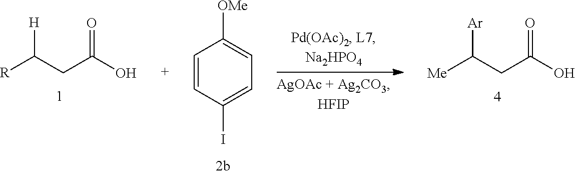

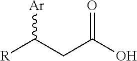

R = Et; 4a, 60%
R = (CH₂)₂CH₃; 4b, 63%
R = (CH₂)₃CH₃; 4c, 62%
R = (CH₂)₄CH₃; 4d, 65%

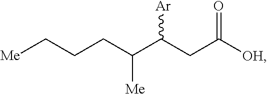

4e, 56%, dr = 1.0

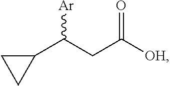

4f, 44%

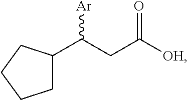

4g, 47%

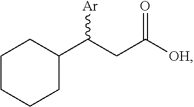

4h, 40%

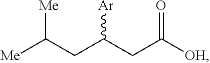

4i, 56%

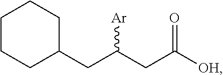

4j, 58%

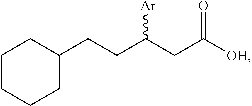

4k, 59%

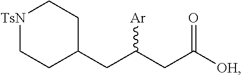

4l, 52%

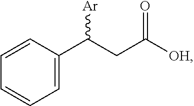

4m, 44%

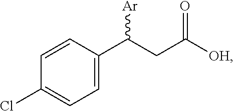

4n, 43%

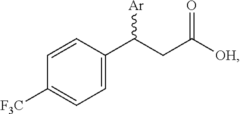

4o, 40%

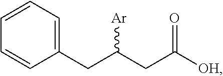

4p, 54%

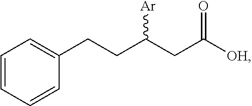

4q, 56%

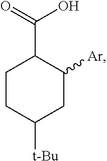

4r, 55%^{d}

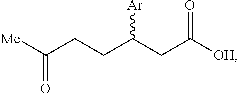

4s, 42%^{c,e}

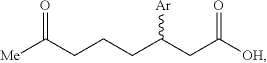

4t, 47%^{c,e}

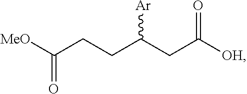

4u, 45%^{e}

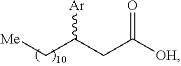

4v, 72%

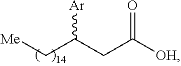

4w, 68%

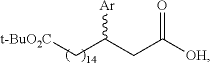

4x, 54%

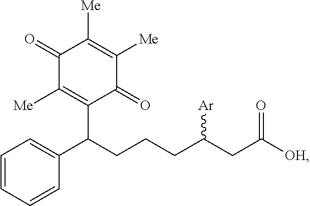

Seratrodast
4y, 52%^{e}, dr = 1.4
^{a}Conditions: 1 (0.1 mmol), aryl iodide 2 (0.2 mmol), Pd(OAc)₂ (10 mol %), L7 (12 mol %), Na₂HPO₄ (1.0 equiv.), AgOAc (2.0 equiv.), Ag₂CO₃ (0.5 equiv.), HFIP (1.0 mL), 100° C., 48 h.
^{b}Isolated yields.
^{c}4-Iodotoluene was used instead of 4-Iodoanisole.
^{d}cis-4-(tert-butyl)cyclohexane carboxylic acid as starting material.
^{e}The reaction was run at 110° C.

branched aliphatic acids, such as naturally occurring caprylic acid and 4-methyloctanoic acid performed well, affording the desired arylated products 4a to 4e in high yields. Substrates containing sterically hindered alkyl groups at the β position were also tolerated, albeit with a slight reduction in the yields of the corresponding products (4f-4h). Acids containing isopropyl, cyclohexyl and cyclohexylmethyl moieties at the γ positions were also successfully arylated to the desired products (4i-4k). In addition, phenyl propanoic acids bearing chloro- and trifluoromethyl-substituents were converted to β-arylated products in high yields (4m-4o). Furthermore, phenylbutyric acid and phenylvaleric acid underwent smooth arylation to provide the corresponding products in good yields (4p-4q). 4-tBu substituted cyclohexane carboxylic acid was also viable substrate (4r). Notably, the β-methylene arylation reaction was also found to be selective for carboxylic acids in the presence of enolizable ketones and esters, affording the desired products in moderate yields (4r-4u). Fatty acids were also found to be compatible with this transformation, providing the β-arylated products in up to 72% yield (4v-4x). To illustrate the utility of this transformation, the antiasthmatic drug seratrodast was subjected to the standard reaction conditions, delivering the desired product in 52% yield (4y).

In summary, as disclosed herein, the first protocol for Pd(II)-catalyzed β-methylene C(sp³)-H arylation of free aliphatic acids without requiring the use of an exogenous directing group has been developed. The key to the success of this method was the use of a bidentate pyridine-pyridone ligand. This new methodology affords a novel disconnection for preparing diverse arylated carboxylic acids from simple starting materials, providing a significant solution to a heretofore unmet need in the field of synthetic organic chemistry.

### REFERENCES

(1) For selected reviews on C—H bond functionalization, see: (a) Lyons, T. W.; Sanford, M. S. Palladium-catalyzed ligand-directed C—H functionalization reactions. Chem. Rev. 2010, 110, 1147-1169. (b) Ackermann, L. Carboxylate-assisted transition-metal-catalyzed C—H bond functionalizations: mechanism and scope. Chem. Rev. 2011, 111, 1315-1345. (c) Daugulis, O.; Roane, J.; Tran, L. D. Bidentate, monoanionic auxiliary-directed functionalization of carbon-hydrogen bonds. Acc. Chem. Res. 2015, 48, 1053-1064. (d) He, J.; Wasa, M.; Chan, K. S. L.; Shao, Q.; Yu, J.-Q. Palladium-catalyzed transformations of alkyl C—H bonds. Chem. Rev. 2017, 117, 8754-8786. (e) Lam, N. Y. S.; Wu, K.; Yu, J.-Q. Advancing the logic of chemical synthesis: C—H activation as strategic and tactical disconnections for C—C bond construction. Angew. Chem. Int. Ed. 2021, 60, 15767-15790.
(2) Giri, R.; Chen, X.; Yu, J.-Q. Palladium-catalyzed asymmetric iodination of unactivated C—H bonds under mild conditions. Angew. Chem. Int. Ed. 2005, 44, 2112-2115.
(3) For selected examples of the application of C(sp³)-H arylation in the total synthesis of natural products and bioactive molecules, see: (a) Feng, Y.; Chen, G. Total synthesis of celogentin C by stereoselective C—H activation. Angew. Chem. Int. Ed. 2010, 49, 958-961. (b) Gutekunst, W. R.; Baran, P. S. Total synthesis and structural revision of the piperarborenines via sequential cyclobutane C—H arylation. J. Am. Chem. Soc. 2011, 133, 19076-19079. (c) Gutekunst, W. R.; Gianatassio, R.; Baran, P. S. Sequential C(sp³)-H arylation and olefination: total synthesis of the proposed structure of pipercyclobutanamide A. Angew. Chem. Int. Ed. 2012, 51, 7507-7510. (d) Ting, C. P.; Maimone, T. J. C—H bond arylation in the synthesis of aryltetralin lignans: A short total synthesis of podophyllotoxin. Angew. Chem. Int. Ed. 2014, 53, 3115-3119. (e) Dailler, D.; Danoun, G.; Baudoin, O. A general and scalable synthesis of aeruginosin marine natural products based on two strategic C(sp3)-H activation reactions. Angew. Chem. Int. Ed. 2015, 54, 4919-4922. (f) Beck, J. C.; Lacker, C. R.; Chapman, L. M.; Reisman, S. E. A modular approach to prepare enantioenriched cyclobutanes: synthesis of (+)-rumphellaone A. Chem. Sci. 2019, 10, 2315-2319. For a review, see: (g) Lucas, E. L.; Lam, N. Y. S.; Zhuang, Z.; Chan, H. S. S.; Strassfeld, D. A.; Yu, J.-Q. Palladium-catalyzed enantioselective β-C(sp3)-H activation reactions of aliphatic acids: a retrosynthetic surrogate for enolate alkylation and conjugate addition. Acc. Chem. Res. 2022, 55, 537-550.
(4) (a) Zaitsev, V. G.; Shabashov, D.; Daugulis, O. Highly regioselective arylation of sp³ C—H bonds catalyzed by palladium acetate. J. Am. Chem. Soc. 2005, 127, 13154-13155. (b) Reddy, B. V. S.; Reddy, L. R.; Corey, E. J. Novel acetoxylation and C—C coupling reactions at unactivated positions in α-amino acid derivatives. Org. Lett. 2006, 8, 3391-3394. (c) Zhang, Q.; Yin, X.-S.; Zhao, S.; Fang, S.-L.; Shi, B.-F. Pd(II)-catalyzed arylation of unactivated methylene C(sp³)-H bonds with aryl halides using a removable auxiliary. Chem. Commun. 2014, 50, 8353-8355. (d) Wei, Y.; Tang, H.; Cong, X.; Rao, B.; Wu, C.; Zeng, X. Pd(II)-catalyzed intermolecular arylation of unactivated C(sp³)-H bonds with aryl bromides enabled by 8-aminoquinoline auxiliary. Org. Lett. 2014, 16, 2248-2251. (e) Kim, J.; Sim, M.; Kim, N.; Hong, S. Asymmetric C—H functionalization of cyclopropanes using an isoleucine-NH₂ bidentate directing group. Chem. Sci. 2015, 6, 3611-3616. (f) Liu, J.; Xie, Y.; Zeng, W.; Lin, D.; Deng, Y.; Lu, X. Pd(II)-catalyzed pyridine N-oxides directed arylation of unactivated C(sp³)-H bonds. J. Org. Chem. 2015, 80, 4618-4626. (g) Reddy, C.; Bisht, N.; Parella, R.; Babu. S. A. 4-Amino-2,1,3-benzothiadiazole as a removable bidentate directing group for the Pd(II)-catalyzed arylation/oxygenation of sp²/sp³ β-C—H bonds of carboxamides. J. Org. Chem. 2016, 81, 12143-12168. (h) Yan, S.-B; Zhang, S.; Duan, W.-L. Palladium-catalyzed asymmetric arylation of C(sp³)-H bonds of aliphatic amides: controlling enantioselectivity using chiral phosphoric amides/acids. Org. Lett. 2015, 17, 2458-2461.
(5) (a) Wasa, M.; Chan, K. S. L.; Zhang, X.-G.; He, J.; Miura, M.; Yu, J.-Q. Ligand-enabled methylene C(sp³)-H bond activation with a Pd(II) catalyst. J. Am. Chem. Soc. 2012, 134, 18570-18572. (b) He, J.; Li, S.; Deng, Y.; Fu, H.; Laforteza, B. N.; Spangler, J. E.; Homs, A.; Yu, J.-Q. Ligand-controlled C(sp³)-H arylation and olefination in synthesis of unnatural chiral α-amino acids. Science 2014, 343, 1216-1220. (c) Xiao, K.-J.; Lin, D. W.; Miura, M. Zhu, R.-Y. Gong, W.; Wasa, M.; Yu, J.-Q. Palladium(II)-catalyzed enantioselective C(sp³)-H activation using a chiral hydroxamic acid ligand. J. Am. Chem. Soc. 2014, 136, 8138-8142. (d) Chen, G.; Gong, W.; Zhuang, Z.; Andra, M. S.; Chen, Y.-Q.; Hong, X.; Yang, Y.-F.; Liu, T.; Houk, K. N.; Yu, J.-Q. Ligand-accelerated enantioselective methylene C(sp3)-H bond activation. Science 2016, 353, 1023-1027.
(6) Engle, K. M.; Mei, T.-S.; Wasa, M.; Yu, J.-Q. Weak coordination as a powerful means for developing broadly useful C—H functionalization reactions. Acc. Chem. Res. 2012, 45, 788-802.
(7) For C(sp³)-H functionalization reactions of free carboxylic acids, see: (a) Giri, R.; Maugel, N.; Li, J.-J.; Wang, D.-H.; Breazzano, S. P.; Saunder, L. B.; Yu, J.-Q. Palladium-catalyzed methylation and arylation of sp² and sp³ C—H bonds in simple carboxylic acids. J. Am. Chem. Soc. 2007, 129, 3510-3511. (b) Chen, G.; Zhuang, Z.; Li, G.-C; Saint-Denis, T. G.; Hsiao, Y.; Joe, C. L.; Yu, J.-Q. Ligand-enabled β-C—H arylation of α-amino acids without installing exogenous directing groups. Angew. Chem., Int. Ed. 2017, 56, 1506-1509. (c) Zhu, Y.; Chen, X.; Yuan, C.; Li, G.; Zhang, J.; Zhao, Y. Pd-catalysed ligand-enabled carboxylate-directed highly regioselective arylation of aliphatic acids. Nat. Commun. 2017, 8, 14904-14101. (d) Ghosh, K. K.; van Gemmeren, M. Pd-catalyzed β-C(sp³)-H arylation of propionic acid and related aliphatic acids. Chem.-Eur. J. 2017, 23, 17697-17700. (e) Shen, P.-X.; Hu, L.; Shao, Q.; Hong, K.; Yu, J.-Q. Pd(II)-catalyzed enantioselective C(sp³)-H arylation of free carboxylic acids. J. Am. Chem. Soc. 2018, 140, 6545-6549. (f) Hu, L.; Shen, P.-X.; Shao, Q.; Hong, K.; Qiao, J. X.; Yu, J.-Q. PdII-catalyzed enantioselective C(sp³)-H activation/cross-coupling reactions of free carboxylic acids. Angew. Chem., Int. Ed. 2019, 58, 2134-2138. (g) Zhuang, Z.; Yu, C.-B.; Chen, G.; Wu, Q.-F.; Hsiao, Y.; Joe, C. L.; Qiao, J. X.; Poss, M. A.; Yu, J.-Q. Ligand-enabled β-C(sp³)-H olefination of free carboxylic acids. J. Am. Chem. Soc. 2018, 140, 10363-10367. (h) Ghosh, K. K.; Uttry, A.; Koldemir, A.; Ong, M.; van Gemmeren, M. Direct β-C(sp³)-H acetoxylation of aliphatic carboxylic acids. Org. Lett. 2019, 21, 7154-7157. (i) Zhuang, Z.; Yu, J.-Q. Lactonization as a general route to β-C(sp³)-H functionalization. Nature 2020, 577, 656-659. (j) Zhuang, Z.; Herron, A. N.; Fan, Z.; Yu, J.-Q. Ligand-enabled monoselective β-C(sp³)-H acyloxylation of free carboxylic acids using a practical oxidant. J. Am. Chem. Soc. 2020, 142, 6769-6776. (k) Ghiringhelli, F.; Uttry, A.; Ghosh, K. K.; van Gemmeren, M. Direct β- and γ-C(sp³)-H alkynylation of free carboxylic acids. Angew. Chem., Int. Ed. 2020, 59, 23127-23131. (1) Zhuang, Z.; Herron, A. N.; Liu, S.; Yu, J.-Q. Rapid construction of tetralin, chromane, and indane motifs via cyclative C—H/C—H coupling: four-step total synthesis of (±)-russujaponol F. J. Am. Chem. Soc. 2021, 143, 687-692. (m) Zhuang, Z.; Herron, A. N.; Yu, J.-Q. Syntheses of cyclic anhydrides via ligand-enabled C—H carbonylation of simple aliphatic acids. Angew. Chem. Int. Ed. 2021, 60, 16382-16387. (n) Uttry, A.; Mal, S.; van Gemmeren, M. Late-stage β-C(sp3)-H deuteration of carboxylic acids. J. Am. Chem. Soc. 2021, 143, 10895-10901. (o) Sheng, T.; Zhuang, Z.; Wang, Z.; Hu, L.; Herron, A. N.; Qiao, J. X.; Yu, J.-Q. One-step synthesis of β-alkylidene-γ-lactones via ligand-enabled β,γ-dehydrogenation of aliphatic acids. J. Am. Chem. Soc. 2022, 144, 12924-12933.
(8) Wang, Z.; Hu, L.; Chekshin, N.; Zhuang, Z.; Qian, S.; Qiao, J. X.; Yu, J.-Q. Ligand-controlled divergent dehydrogenative reactions of carboxylic acids via C—H activation. Science 2021, 374, 1281-1285.
(9) Chan, H. S. S.; Yang, J.-M.; Yu, J.-Q. Catalyst-controlled site-selective methylene C—H lactonization of dicarboxylic acids. Science 2022, 376, 1481-1487.

### EMBODIMENTS

The application provides the following Embodiments:

Embodiment 1. A method of β-methylene C(sp³)-H arylation of aliphatic carboxylic acids, comprising treating an aliphatic carboxylic acid with an aryl iodide in the presence of a bidentate pyridine-pyridone ligand (L) and a Pd(II) catalyst.

Embodiment 2. The method of Embodiment 1, wherein the method of β-methylene C(sp³)-H arylation of aliphatic carboxylic acids occurs according to the following reaction scheme:

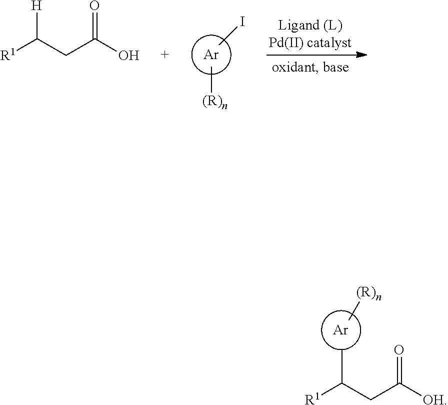

wherein:
Ar is (C₆-C₁₀)aryl;
R is H, halo, OH, (C₁-C₆)alkyl, halo (C₁-C₆)alkyl, (C₁-C₆)heteroalkyl, halo (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, Ac, —OAc, —NO₂, Ts, —OTBS, —C(═O)H, NH₂, —C(═O)C₁-C₆)alkyl, —C(═O)O(C₁-C₆)alkyl, oxo, —S(═O)(C₁-C₆)alkyl, —S(═O)₂(C₁-C₆)alkyl, or —C(═O)NH(C₁-C₆)alkyl;
R¹ is H or selected from the group consisting of (C₁-C₁₂)alkyl, (C₂₋₁₂)alkenyl, (C₂₋₁₂)alkynyl, (C₁-C₁₀)heteroalkyl, hetero (C₂₋₁₀)alkenyl, hetero (C₂₋₁₀)alkynyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, each independently and optionally substituted with one or more R¹′;
   each R¹′ is independently OH, halo, CN, (C₁-C₆)alkyl, halo (C₁-C₆)alkyl, (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, NH₂, —C(═O)(C₁-C₆)alkyl, —C(═O)O(C₁-C₆)alkyl, oxo, Ts, —S(═O)₂F, —OS(═O)₂F, or —S(═O)₂(C₁-C₆)alkyl; and
n is 0, 1, 2, or 3.

Embodiment 3. The method of Embodiment 2, wherein Ar is Ph.

Embodiment 4. The method of either Embodiment 2 or Embodiment 3, wherein n is 0.

Embodiment 5. The method of either Embodiment 2 or Embodiment 3, wherein n is 1.

Embodiment 6. The method of Embodiment 5, wherein R is (C₁-C₆)alkyl.

Embodiment 7. The method of Embodiment 5, wherein R is Me.

Embodiment 8. The method of Embodiment 5, wherein R is halo.

Embodiment 9. The method of Embodiment 5, wherein R is CF₃.

Embodiment 10. The method of Embodiment 5, wherein R is —OMe.

Embodiment 11. The method of Embodiment 5, wherein R is Ac.

Embodiment 12. The method of Embodiment 5, wherein R is Ph.

Embodiment 13. The method of Embodiment 5, wherein R is —C(═O)OMe.

Embodiment 14. The method of Embodiment 5, wherein R is NO₂.

Embodiment 15. The method of Embodiment 5, wherein R is Ts.

Embodiment 16. The method of Embodiment 5, wherein R is —OTBS.

Embodiment 17. The method of any one of Embodiments 2-16, wherein R¹ is (C₁-C₁₂)alkyl optionally substituted with one or more R¹′.

Embodiment 18. The method of Embodiment 17, wherein R¹ is (C₁-C₆)alkyl.

Embodiment 19. The method of Embodiment 17 or Embodiment 18, wherein R¹′ is —O(C₁-C₆)alkyl.

Embodiment 20. The method of Embodiment 17 or Embodiment 18, wherein R¹′ is —C(═O)O(C₁-C₆)alkyl.

Embodiment 21. The method of Embodiment 17 or Embodiment 18, wherein R¹′ is —C(═O)(C₁-C₆)alkyl.

Embodiment 22. The method of Embodiment 17 or Embodiment 18, wherein R¹′ is halo.

Embodiment 23. The method of any one of Embodiments 2-16, wherein R¹ is (C₁-C₁₂)heteroalkyl optionally substituted with one or more R¹′.

Embodiment 24. The method of any one of Embodiments 2-16, wherein R¹ is (C₆-C₁₀)aryl optionally substituted with one or more R¹′.

Embodiment 25. The method of Embodiment 24, wherein R¹ is Ph optionally substituted with one or more R¹′.

Embodiment 26. The method of any one of Embodiments 2-16, wherein R¹ is (C₁-C₆)alkyl (C₆-C₁₀)aryl optionally substituted with one or more R¹′.

Embodiment 27. The method of Embodiment 26, wherein R¹ is Bn optionally substituted with one or more R¹′.

Embodiment 28. The method of any one of Embodiments 2-16, wherein R¹ is (C₃-C₇)cycloalkyl optionally substituted with one or more R¹′.

Embodiment 29. The method of any one of Embodiments 2-16, wherein R¹ is (C₁-C₆)alkyl (C₃-C₇)cycloalkyl optionally substituted with one or more R¹′.

Embodiment 30. The method of any one of Embodiments 23-29, wherein R¹′ is (C₁-C₆)alkyl, —OMe, CF₃, or halo.

Embodiment 31. The method of Embodiment 2, wherein the method of β-methylene C(sp³)-H arylation of aliphatic carboxylic acids occurs according to the following reaction scheme:

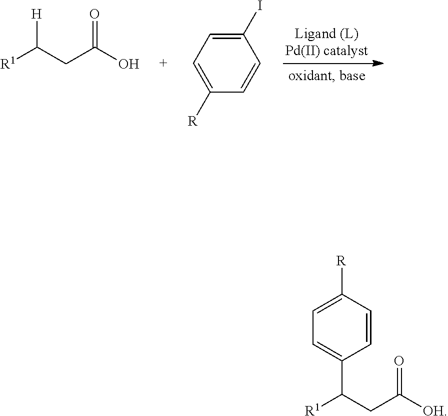

Embodiment 32. The method of Embodiment 31, wherein R is (C₁-C₆)alkyl.

Embodiment 33. The method of Embodiment 32, wherein R is Me.

Embodiment 34. The method of Embodiment 31, wherein R is F.

Embodiment 35. The method of Embodiment 31, wherein R is Cl.

Embodiment 36. The method of Embodiment 31, wherein R is Br.

Embodiment 37. The method of Embodiment 31, wherein R is —OMe.

Embodiment 38. The method of Embodiment 31, wherein R is CF₃.

Embodiment 39. The method of Embodiment 31, wherein R is Ph.

Embodiment 40. The method of Embodiment 31, wherein R is NO₂.

Embodiment 41. The method of Embodiment 31, wherein R is —OAc.

Embodiment 42. The method of Embodiment 31, wherein R is —C(═O)OMe.

Embodiment 43. The method of Embodiment 31, wherein R is —OTBS.

Embodiment 44. The method of any one of Embodiments 31-43, wherein R¹ is (C₁-C₁₂)alkyl optionally substituted with one or more R¹′.

Embodiment 45. The method of Embodiment 44, wherein R¹′ is —C(═O)O(C₁-C₆)alkyl.

Embodiment 46. The method of Embodiment 44, wherein R¹′ is —C(═O)(C₁-C₆)alkyl.

Embodiment 47. The method of any one of Embodiments 31-43, wherein R¹ is (C₁-C₁₂)heteroalkyl optionally substituted with one or more R¹′.

Embodiment 48. The method of any one of Embodiments 31-43, wherein R¹ is (C₆-C₁₀)aryl optionally substituted with one or more R¹′.

Embodiment 49. The method of Embodiment 48, wherein R¹ is Ph optionally substituted with one or more R¹′.

Embodiment 50. The method of Embodiment 49, wherein R¹′ is (C₁-C₆)alkyl.

Embodiment 51. The method of Embodiment 49, wherein R¹′ is halo.

Embodiment 52. The method of Embodiment 49, wherein R¹′ is —C(═O)O(C₁-C₆)alkyl or —C(═O)(C₁-C₆)alkyl.

Embodiment 53. The method of Embodiment 49, wherein R¹′ is Me, F, Cl, Br, —OMe, CF₃, —C(═O)Me, or —C(═O)Ot-Bu.

Embodiment 54. The method of any one of Embodiments 31-43, wherein R¹ is (C₁-C₆)alkyl (C₆-C₁₀)aryl optionally substituted with one or more R¹′.

Embodiment 55. The method of Embodiment 54, wherein R¹ is Bn optionally substituted with one or more R¹′.

Embodiment 56. The method of any one of Embodiments 31-43, wherein R¹ is (C₃-C₇)cycloalkyl optionally substituted with one or more R¹′.

Embodiment 57. The method of any one of Embodiments 31-43, wherein R¹ is (C₁-C₆)alkyl (C₃-C₇)cycloalkyl, optionally substituted with one or more R¹′.

Embodiment 58. The method of any one of Embodiments 31-43, wherein R¹ is (C₁-C₆)alkyl (C₃-C₇)heterocycloalkyl optionally substituted with one or more R¹′.

Embodiment 59. The method of any one of Embodiments 54-58, wherein R¹′ is (C₁-C₆)alkyl, halo, —O(C₁-C₆)alkyl, CF₃, —C(═O)(C₁-C₆)alkyl, or —C(═O)O(C₁-C₆)alkyl.

Embodiment 60. The method of any one of Embodiments 54-59, wherein R¹′ is Me, F, Cl, Br, —OMe, CF₃, —C(═O)Me, or —C(═O)Ot-Bu.

Embodiment 61. The method any one of Embodiments 2-60, wherein the Pd(II) catalyst is Pd(OAc)₂.

Embodiment 62. The method any one of Embodiments 2-61, wherein the base is Na₂HPO₄.

Embodiment 63. The method any one of Embodiments 2-62, wherein the solvent is HFIP.

Embodiment 64. The method any one of Embodiments 2-63, wherein the oxidant is a mixture of AgOAc and Ag₂CO₃.

Embodiment 65. The method any one of Embodiments 2-63, wherein the oxidant is AgOAc.

Embodiment 66. The method any one of Embodiments 2-63, wherein the oxidant is Ag₂CO₃.

Embodiment 67. The method any one of Embodiments 2-66, wherein the reaction temperature is approximately 80-120° C.

Embodiment 68. The method of Embodiment 67, wherein the reaction temperature is approximately 100° C.

Embodiment 69. The method of any one of Embodiments 1-68, wherein L is selected from the group consisting of:

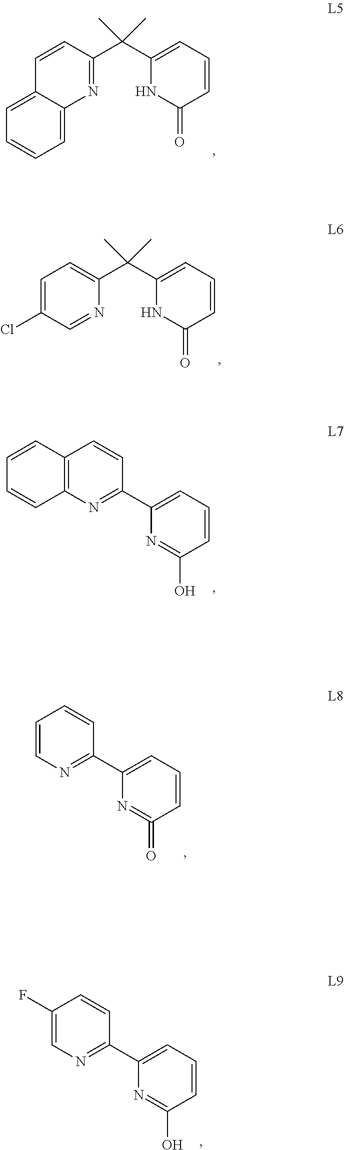

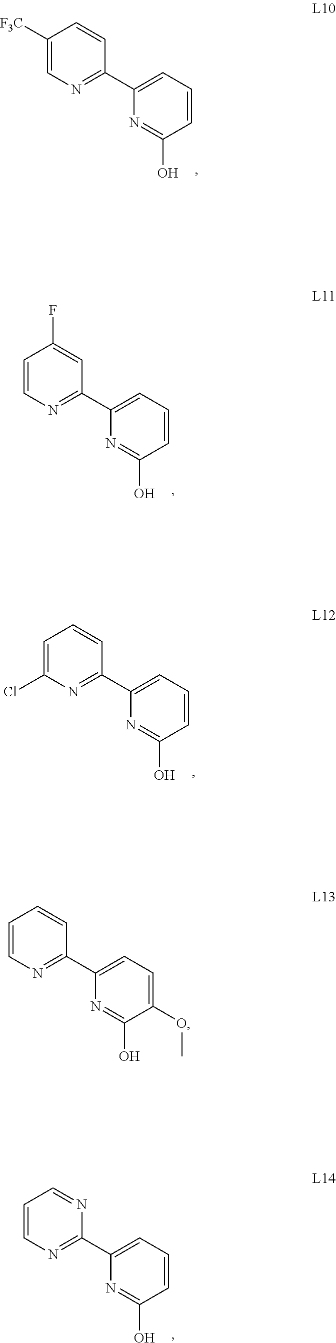 [SMILES: Oc1cccc(-c2ccc(C(F)(F)F)cn2)n1 ; Oc1cccc(-c2cc(F)ccn2)n1 ; Oc1cccc(-c2cccc(Cl)n2)n1 ; COc1ccc(-c2ccccn2)nc1O ; Oc1cccc(-c2ncccn2)n1]

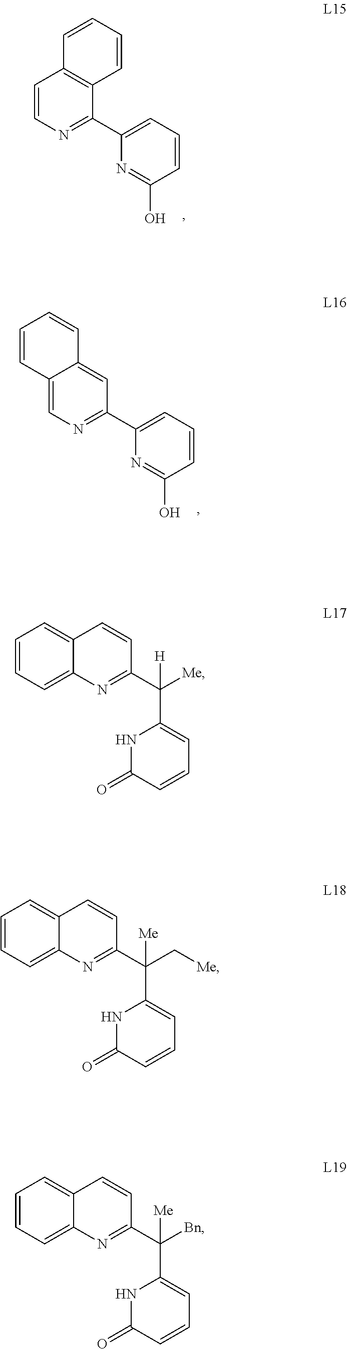 [SMILES: Oc1cccc(-c2nccc3ccccc23)n1 ; Oc1cccc(-c2cc3ccccc3cn2)n1 ; CC(c1ccc2ccccc2n1)c1cccc(=O)[nH]1 ; CCC(C)(c1ccc2ccccc2n1)c1cccc(=O)[nH]1 ; CC(Cc1ccccc1)(c1ccc2ccccc2n1)c1cccc(=O)[nH]1]

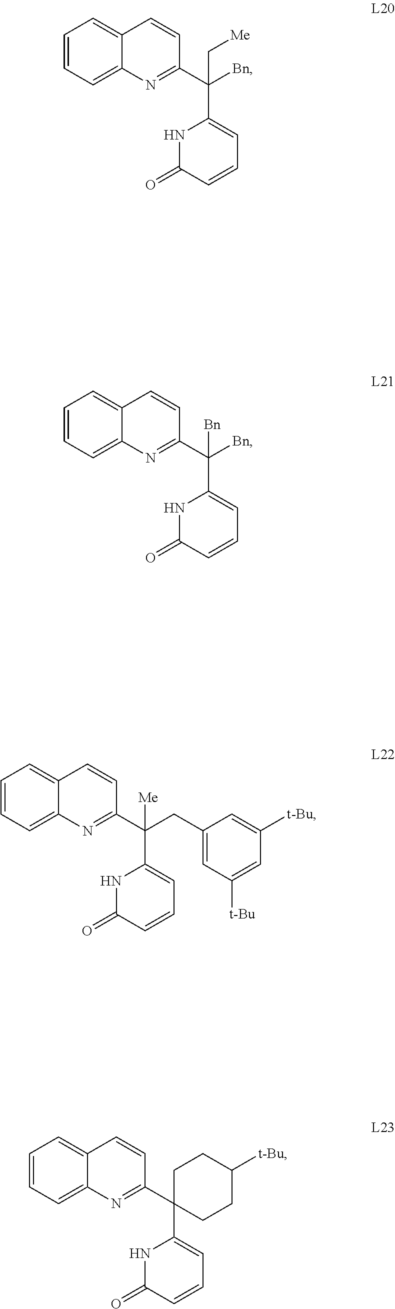 [SMILES: CCC(Cc1ccccc1)(c1ccc2ccccc2n1)c1cccc(=O)[nH]1 ; O=c1cccc(C(Cc2ccccc2)(Cc2ccccc2)c2ccc3ccccc3n2)[nH]1 ; CC(C)(C)c1cc(CC(C)(c2ccc3ccccc3n2)c2cccc(=O)[nH]2)cc(C(C)(C)C)c1 ; CC(C)(C)C1CCC(c2ccc3ccccc3n2)(c2cccc(=O)[nH]2)CC1]

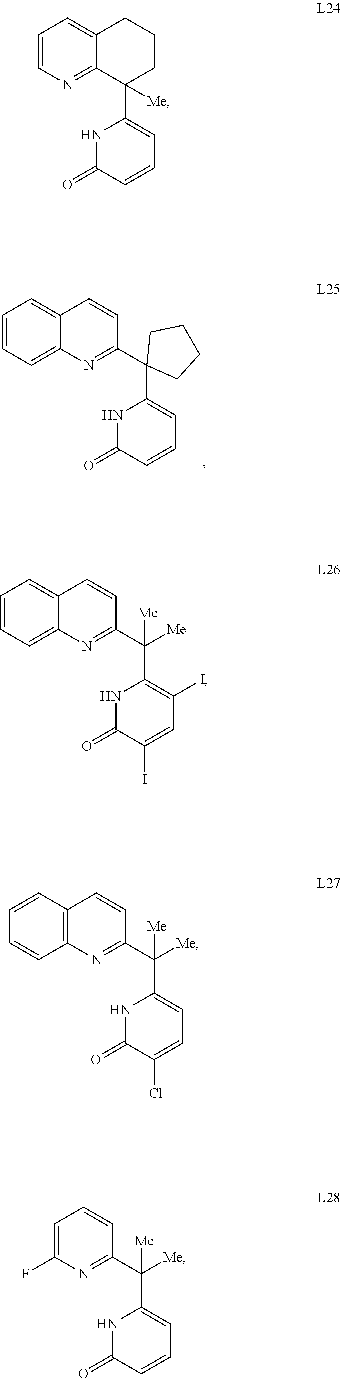 [SMILES: CC1(c2cccc(=O)[nH]2)CCCc2cccnc21 ; O=c1cccc(C2(c3ccc4ccccc4n3)CCCC2)[nH]1 ; CC(C)(c1ccc2ccccc2n1)c1[nH]c(=O)c(I)cc1I ; CC(C)(c1ccc2ccccc2n1)c1ccc(Cl)c(=O)[nH]1 ; CC(C)(c1cccc(F)n1)c1cccc(=O)[nH]1]

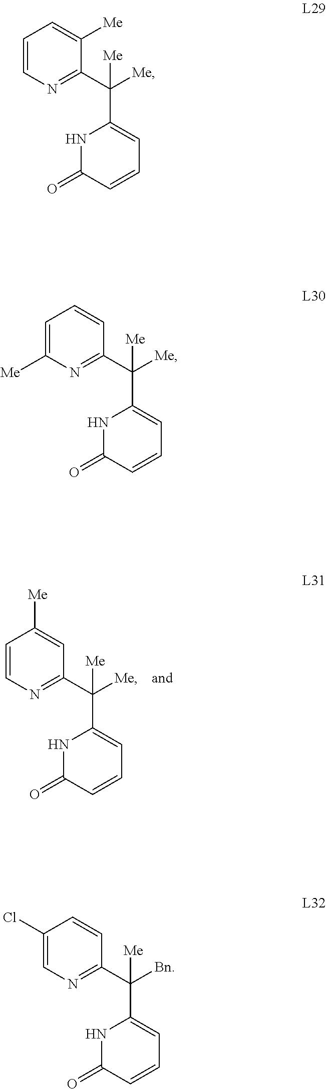 [SMILES: Cc1cccnc1C(C)(C)c1cccc(=O)[nH]1 ; Cc1cccc(C(C)(C)c2cccc(=O)[nH]2)n1 ; Cc1ccnc(C(C)(C)c2cccc(=O)[nH]2)c1 ; CC(Cc1ccccc1)(c1ccc(Cl)cn1)c1cccc(=O)[nH]1]

Embodiment 70. The method of Embodiment 69, wherein L is L7.

Embodiment 71. The method of Embodiment 2, wherein the method of β-methylene C(sp³)-H arylation of aliphatic carboxylic acids occurs according to the following reaction scheme:

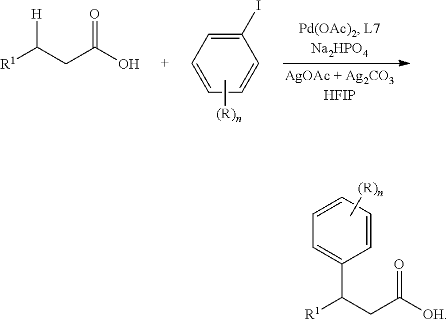

Embodiment 72. The method of Embodiment 71, wherein n is 0.

Embodiment 73. The method of Embodiment 71, wherein n is 1.

Embodiment 74. The method of Embodiment 71, wherein n is 2.

Embodiment 75. The method of any one of Embodiments 71-74, wherein R¹ is (C₁-C₁₂)alkyl optionally substituted with one or more R¹′.

Embodiment 76. The method of any one of Embodiments 71-75, wherein R¹′ is Me, F, Cl, Br, —OMe, CF₃, —C(═O)Me, or —C(═O)Ot-Bu.

Embodiment 77. Any method of β-methylene C(sp³)-H arylation of aliphatic carboxylic acids as disclosed herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “˜˜˜˜” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertible species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiazoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂,C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the an, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### 1. General Information

Pd(OAc)₂ was purchased from Sigma-Aldrich. Ag₂CO₃ were purchased from Strem. Solvents were obtained from Sigma-Aldrich, Acros and Oakwood, and used directly without further purification. Aryl iodides were obtained from the commercial sources. Carboxylic acids were obtained from the commercial sources or synthesized following literature procedures. Other reagents were purchased at the highest commercial quality and used without further purification, unless otherwise stated. Analytical thin layer chromatography was performed on 0.25 mm silica gel 60-F254. Visualization was carried out with UV light. ¹H NMR spectra were recorded on Bruker DRX-600 instrument. Chemical shifts were quoted in parts per million (ppm) referenced to 0.00 ppm for TMS. The following abbreviations (or combinations thereof) were used to explain multiplicities: s=singlet, d=doublet, t=triplet, q=quartet, m=multiplet, br=broad. Coupling constants, J, were reported in Hertz unit (Hz). 13C NMR spectra were recorded on Bruker DRX-600 was fully decoupled by broad band proton decoupling. Chemical shifts were reported in ppm referenced to the centre line of a triplet at 77.16 ppm of CDCl₃. ¹⁹F NMR spectra were recorded on Bruker AMX-400 instrument (376 MHz) and were fully decoupled by broad band proton decoupling. Chemical shifts were reported in ppm referenced to the centre line of a triplet at 77.0 ppm of chloroform-d or the centre line of a heptet at 49.0 ppm. Column chromatography was performed using E. Merck silica (60, particle size 0.043-0.063 mm), and preparative thin layer chromatography (pTLC) was performed on Merck silica plates (60F-254). High-resolution mass spectra (HRMS) were recorded on an Agilent Mass spectrometer using ESI-TOF (electrospray ionization-time of flight).

### 2. Scope of Aliphatic Carboxylic Acids

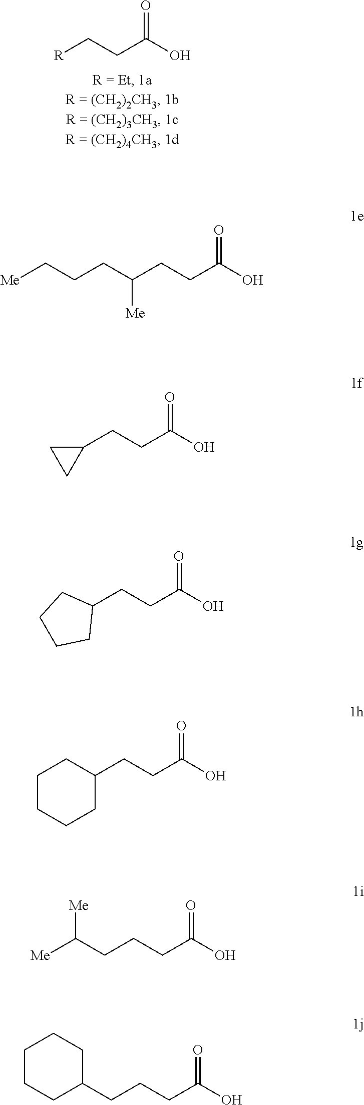

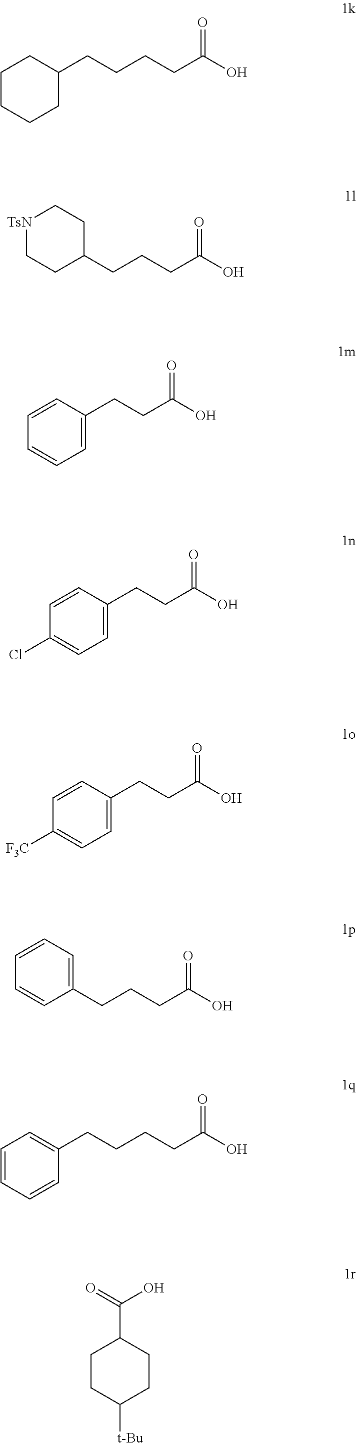

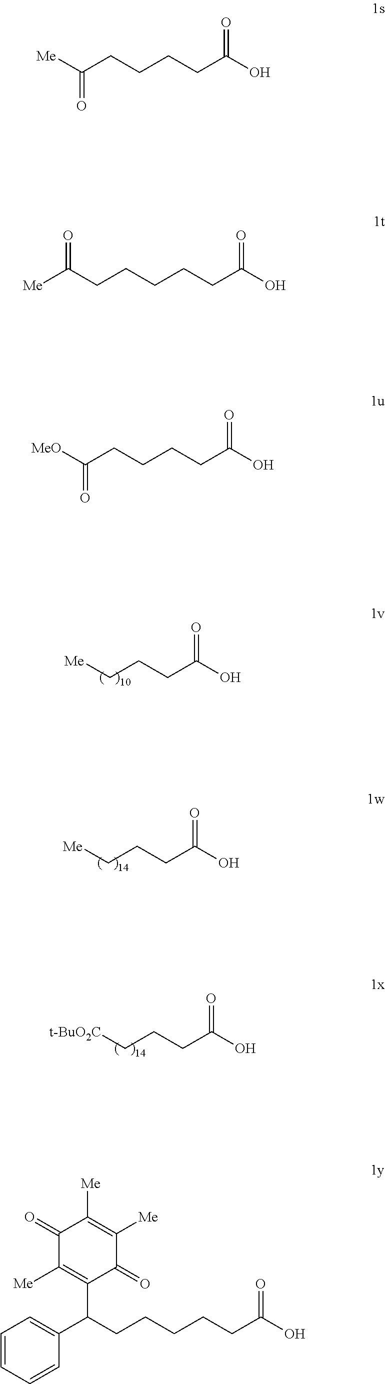

Aliphatic carboxylic acids were obtained from the commercial sources.

### 3. Experimental Section

### 3.1 Optimization of Conditions of β-Methylene C(sp³)-H Arylation of Free Aliphatic Acids

TABLE S1
Screening of Ag Salts and Pd Source^{a,b}

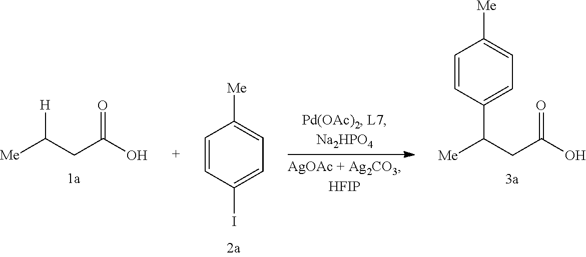 [SMILES: CCCC(=O)O ; Cc1ccc(I)cc1 ; Cc1ccc(C(C)CC(=O)O)cc1]

| Oxidant | Yield | Pd source | Yield |
|---|---|---|---|
| no change | 62% | PdCl₂ | 21% |
| Ago | 23% | Pd(TFA)₂ | 12% |
| Ag₂CO₃ | 42% | Pd(MeCN)₄(BF₄)₂ | 18% |
| AgOAc | 50% | Pd(MeCN)₂Cl₂ | 32% |
| AgTFA | 14% | Pd(PhCN)₂Cl₂ | 23% |
| AgNO₃ | 23% | [Pd(allyl)Cl]₂ | 20% |

^{a}Conditions: 1a (0.1 mmol), aryl iodide 2a (0.2 mmol), Pd(OAc)₂ (10 mol %). ligand (12 mol %), Na₂HPO₄ (1.0 equiv), AgOAc (2.0 equiv), Ag₂CO₃ (0.5 equiv), HFIP (1.0 mL), 100° C., 48 h.
^{b1}H NMR yields, using CH₂Br₂ as an internal standard.

TABLE S2
Screening of Solvent and Temperature^{a,b}

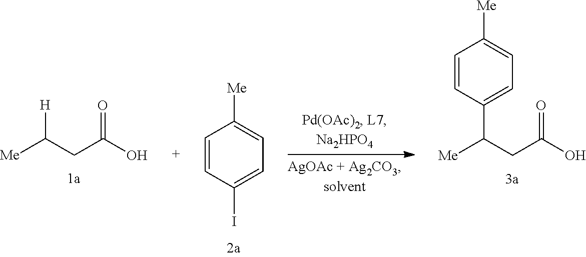 [SMILES: CCCC(=O)O ; Cc1ccc(I)cc1 ; Cc1ccc(C(C)CC(=O)O)cc1]

| Entry | Solvent | Temperature | Yield |
|---|---|---|---|
| 1 | t-BuOH | 100° C. | 18% |
| 2 | MeCN | 100° C. | 9% |
| 3 | THF | 100° C. | trace |
| 4 | Dioxane | 100° C. | 7% |
| 5 | DCE | 100° C. | NR |
| 8 | HFIP | 80° C. | 20% |
| 9 | HFIP | 110° C. | 52% |

^{a}Conditions: 1a (0.1 mmol), aryl iodide 2a (0.2 mmol), Pd(OAc)₂ (10 mol %), ligand (12 mol %), Na₂HPO₄ (1.0 equiv), AgOAc (2.0 equiv), Ag₂CO₃ (0.5 equiv), solevnt (1.0 mL), 100° C. 48 h.
^{b1}H NMR yields, using CH₂Br₂ as an internal standard.

TABLE S3
Further Screening of Ligands^{a,b}

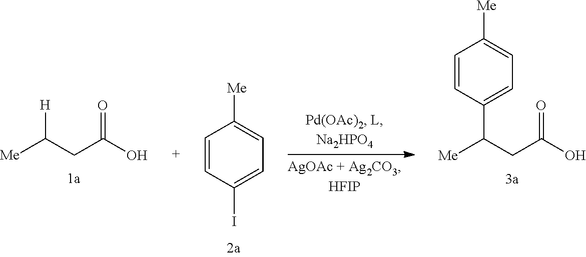 [SMILES: CCCC(=O)O ; Cc1ccc(I)cc1 ; Cc1ccc(C(C)CC(=O)O)cc1]

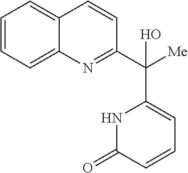 [SMILES: CC(O)(c1ccc2ccccc2n1)c1cccc(=O)[nH]1]

L17
<5%

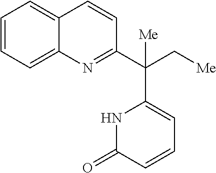 [SMILES: CCC(C)(c1ccc2ccccc2n1)c1cccc(=O)[nH]1]

L18
12%

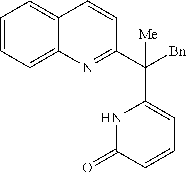 [SMILES: CC(Cc1ccccc1)(c1ccc2ccccc2n1)c1cccc(=O)[nH]1]

L19
19%

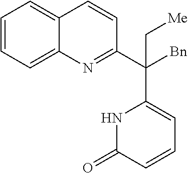 [SMILES: CCC(Cc1ccccc1)(c1ccc2ccccc2n1)c1cccc(=O)[nH]1]

L20
8%

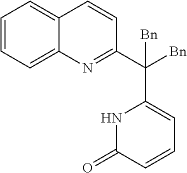 [SMILES: O=c1cccc(C(Cc2ccccc2)(Cc2ccccc2)c2ccc3ccccc3n2)[nH]1]

L21
<5%

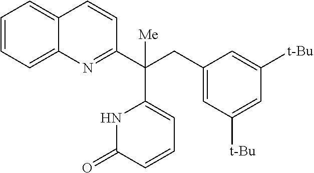 [SMILES: CC(C)(C)c1cc(CC(C)(c2ccc3ccccc3n2)c2cccc(=O)[nH]2)cc(C(C)(C)C)c1]

L22
14%

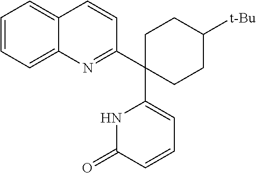 [SMILES: CC(C)(C)C1CCC(c2ccc3ccccc3n2)(c2cccc(=O)[nH]2)CC1]

L23
8%

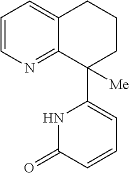 [SMILES: CC1(c2cccc(=O)[nH]2)CCCc2cccnc21]

L24
22%

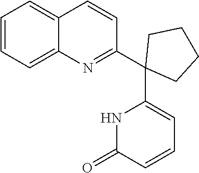 [SMILES: O=c1cccc(C2(c3ccc4ccccc4n3)CCCC2)[nH]1]

L25
13%

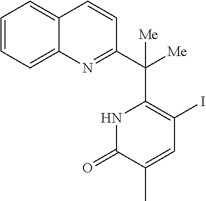 [SMILES: Cc1cc(I)c(C(C)(C)c2ccc3ccccc3n2)[nH]c1=O]

L26
15%

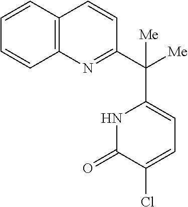 [SMILES: CC(C)(c1ccc2ccccc2n1)c1ccc(Cl)c(=O)[nH]1]

L27
14%

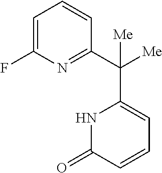 [SMILES: CC(C)(c1cccc(F)n1)c1cccc(=O)[nH]1]

L28
15%

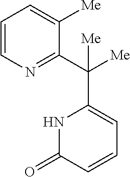 [SMILES: Cc1cccnc1C(C)(C)c1cccc(=O)[nH]1]

L29
12%

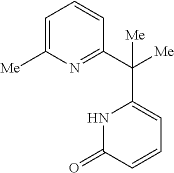 [SMILES: Cc1cccc(C(C)(C)c2cccc(=O)[nH]2)n1]

L30
9%

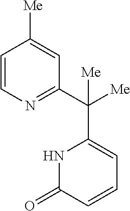 [SMILES: Cc1ccnc(C(C)(C)c2cccc(=O)[nH]2)c1]

L31
12%

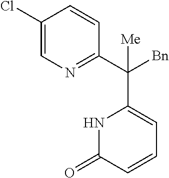 [SMILES: CC(Cc1ccccc1)(c1ccc(Cl)cn1)c1cccc(=O)[nH]1]

L32
10%
^{a}Conditions: 1a (0.1 mmol), aryl iodide 2a (0.2 mmol), Pd(OAc)₂ (10 mol %), ligand (12 mol %), Na₂HPO₄ (1.0 equiv), AgOAc (2.0 equiv), Ag₂CO₃ (0.5 equiv), HFIP (1.0 mL), 100° C., 48 h.
^{b1}H NMR yields, using CH₂Br₂ as an internal standard.

3.2 Substrate Scope of the β-Methylene C(sp³)-H Arylation of Free Aliphatic Acids ^{a,b}

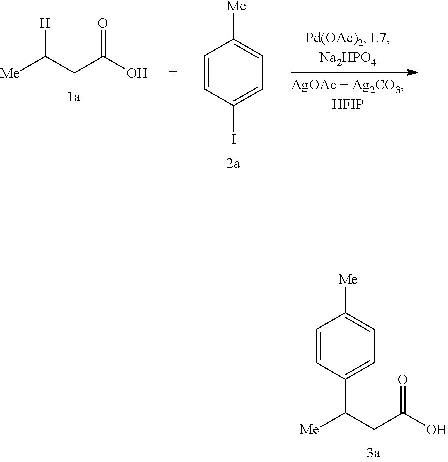 [SMILES: CCCC(=O)O ; Cc1ccc(I)cc1 ; Cc1ccc(C(C)CC(=O)O)cc1]

General procedure for β-methylene C(sp³)-H of free carboxylic acids: Carboxylic acid (0.1 mmol), Ar—I (0.2 mmol), Pd(OAc)₂ (10 mol %), Ligand (12 mol %), Ag₂CO₃ (0.5 equiv.), AgOAc (2.0 equiv.), Na₂HPO₄ (1.0 equiv.) and HFIP (1.0 ml) were added to a reaction vial (8 ml). The vial was capped under air and closed tightly. Then the reaction mixture was stirred at 100° C. for 48 hours. After cooling to room temperature, the mixture was acidified with AcOH (5.0 equiv.) and filtered through a pad of celite with acetone as the eluent to remove the insoluble precipitate. The resulting solution was concentrated and purified by preparative thin-layer chromatography to afford the desired product.

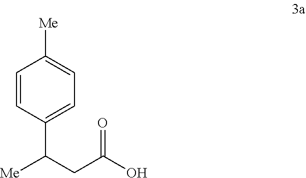 [SMILES: Cc1ccc(C(C)CC(=O)O)cc1]

### 3-(p-tolyl)butanoic Acid (3a)

Substrate 3a was arylated following the general arylated procedure (eluent: hexane/ethyl acetate=5/1). The product was obtained as a white solid (11.0 mg, 62% yield). ¹H NMR (600 MHz, Chloroform-d) δ 7.14 (m, 4H), 3.27 (m, J=8.1, 6.8 Hz, 1H), 2.68 (dd, J=15.5, 6.9 Hz, 1H), 2.59 (dd, J=15.5, 8.2 Hz, 1H), 2.34 (s, 3H), 1.33 (d, J=7.0 Hz, 3H). ¹³C NMR (151 MHz, CDCl₃) δ 177.14, 142.45, 136.03, 129.25, 126.57, 42.42, 35.80, 21.97, 21.01. HRMS (ESI-TOF) Calcd for C₁₁H₁₃O₂ [M−H]⁻: 177.0921. found: 177.0924.

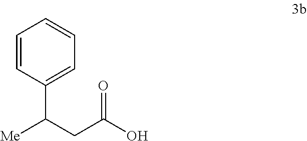 [SMILES: CC(CC(=O)O)c1ccccc1]

### 3-phenylbutanoic Acid (3b)

Substrate 3b was arylated following the general arylated procedure (eluent: hexane/ethyl acetate=5/1). The product was obtained as a white solid (9.3 mg, 57% yield). ¹H NMR (600 MHz, Chloroform-d) δ 7.30 (ddd, J=8.4, 7.0, 1.5 Hz, 2H), 7.24-7.18 (m, 3H), 3.27 (dt, J=8.5, 6.7 Hz, 1H), 2.67 (ddd, J=15.6, 6.7, 1.2 Hz, 1H), 2.58 (ddd, J=15.6, 8.2, 0.9 Hz, 1H), 1.32 (dd, J=7.0, 1.1 Hz, 3H). ¹³C NMR (151 MHz, CDCl₃) δ 178.46, 145.44, 128.59, 126.72, 126.53, 42.55, 36.16, 21.87.

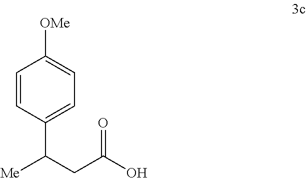 [SMILES: COc1ccc(C(C)CC(=O)O)cc1]

### 3-(4-methoxyphenyl)butanoic Acid (3c)

Substrate 3c was arylated following the general arylated procedure (eluent: hexane/ethyl acetate=3/1). The product was obtained as a white solid (11.6 mg, 60% yield). ¹H NMR (600 MHz, Chloroform-d) δ 7.17 (d, J=8.6 Hz, 2H), 6.87 (d, J=8.7 Hz, 2H), 3.81 (s, 3H), 3.25 (m, 1H), 2.65 (dd, J=15.4, 6.9 Hz, 1H), 2.57 (dd, J=15.4, 8.1 Hz, 1H), 1.32 (d, J=7.0 Hz, 3H). ³C NMR (151 MHz, CDCl₃) δ 177.79, 158.15, 137.58, 127.64, 113.93, 55.25, 42.77, 35.43, 22.03. HRMS (ESI-TOF) Calcd for C₁₁H₁₃O₃[M−H]⁻: 193.0870. found: 193.0870.

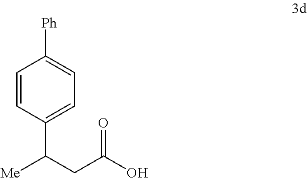 [SMILES: CC(CC(=O)O)c1ccc(-c2ccccc2)cc1]

### 3-([1,1′-biphenyl]-4-yl)butanoic Acid (3d)

Substrate 3d was arylated following the general arylated procedure (eluent: hexane/ethyl acetate=5/1). The product was obtained as a white solid (15.1 mg, 63% yield). ¹H NMR (400 MHz, Chloroform-d) δ 7.61-7.54 (m, 4H), 7.50-7.42 (m, 3H), 7.35-7.31 (m, 2H), 3.36 (m, 1H), 2.75 (dd, J=15.4, 7.1 Hz, 1H), 2.70-2.61 (m, 1H), 1.39 (d, J=6.7 Hz, 3H). ¹³C NMR (151 MHz, CDCl₃) δ 177.40, 144.53, 140.90, 139.47, 128.74, 127.31, 127.16, 127.14, 127.03, 42.37, 35.86, 21.91. HRMS (ESI-TOF) Calcd for C₁₆H₁₅O₂ [M−H]⁻: 239.1077. found: 239.1081.

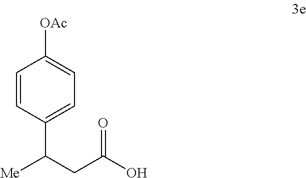 [SMILES: CC(=O)Oc1ccc(C(C)CC(=O)O)cc1]

### 3-(4-acetoxyphenyl)butanoic Acid (3e)

Substrate 3e was arylated following the general arylated procedure (eluent: hexane/ethyl acetate=3/1). The product was obtained as a white solid (11.8 mg, 53% yield). ¹H NMR (600 MHz, Chloroform-d) δ 7.26 (d, J=8.5 Hz, 2H), 7.05 (d, J=8.5 Hz, 2H), 3.31 (m, 1H), 2.69 (dd, J=15.6, 6.9 Hz, 1H), 2.60 (dd, J=15.6, 8.2 Hz, 1H), 2.31 (s, 3H), 1.34 (d, J=7.0 Hz, 3H). ¹³C NMR (151 MHz, CDCl₃) δ 174.80, 169.61, 149.16, 142.95, 127.73, 121.58, 41.99, 35.69, 21.86, 21.15. HRMS (ESI-TOF) Calcd for C₁₂H₁₃O₄ [M−H]⁻: 221.0819. found: 221.0819.

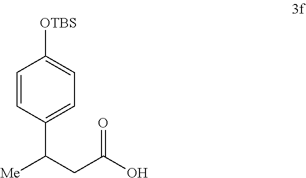 [SMILES: CC(CC(=O)O)c1ccc(O[Si](C)(C)C(C)(C)C)cc1]

### 3-(4-((tert-butyldimethylsilyl)oxy)phenyl)butanoic Acid (3f)

Substrate 3f was arylated following the general arylated procedure (eluent: hexane/ethyl acetate=5/1). The product was obtained as a white solid (16.2 mg, 55% yield). ¹H NMR (400 MHz, Chloroform-d) δ 7.09 (d, J=8.2 Hz, 2H), 6.79 (d, J=8.2 Hz, 2H), 3.24 (m, 1H), 2.65 (dd, J=15.4, 6.6 Hz, 1H), 2.56 (dd, J=16.0, 8.3 Hz, 1H), 1.32 (d, J=7.0 Hz, 3H), 1.00 (s, 9H), 0.21 (s, 6H). ¹³C NMR (151 MHz, CDCl₃) δ 177.55, 154.10, 138.12, 127.54, 119.98, 42.73, 35.47, 25.69, 21.91, 18.18, 0.01. HRMS (ESI-TOF) Calcd for C₁₆H₂₅O₃Si [M−H]⁻: 293.1578. found: 293.1581.

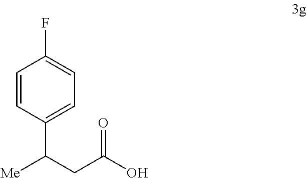 [SMILES: CC(CC(=O)O)c1ccc(F)cc1]

### 3-(4-fluorophenyl)butanoic Acid (3g)

Substrate 3g was arylated following the general arylated procedure (eluent: hexane/ethyl acetate=5/1). The product was obtained as a white solid (9.1 mg, 50% yield). ¹H NMR (600 MHz, Chloroform-d) δ 7.21 (dd, J=8.3, 5.4 Hz, 2H), 7.01 (t, J=8.6 Hz, 2H), 3.29 (m, 1H), 2.63 (m, 2H), 1.33 (d, J=7.0 Hz, 3H). ¹³C NMR (151 MHz, CDCl₃) δ 177.33, 161.51 (d, J=244.6 Hz), 141.02 (d, J=3.0 Hz), 128.14 (d, J=7.6 Hz), 115.33 (d, J=21.0 Hz), 42.54, 35.53, 22.03. ¹⁹F NMR (376 MHz, CDCl₃) δ −119.34. HRMS (ESI-TOF) Calcd for C₁₀H₁₀O₂F [M−H]⁻: 181.0670. found: 181.0666.

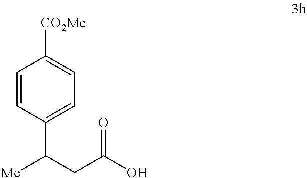 [SMILES: COC(=O)c1ccc(C(C)CC(=O)O)cc1]

### 3-(4-(methoxycarbonyl)phenyl)butanoic Acid (3h)

Substrate 3h was arylated following the general arylated procedure (eluent: hexane/ethyl acetate=3/1). The product was obtained as a white solid (11.8 mg, 53% yield). ¹H NMR (400 MHz, Chloroform-d) δ 8.00 (d, J=8.4 Hz, 2H), 7.32 (d, J=8.2 Hz, 2H), 3.93 (s, 3H), 3.36 (m, 1H), 2.75-2.59 (m, 2H), 1.36 (d, J=7.0 Hz, 3H). ¹³C NMR (151 MHz, CDCl₃) δ 176.60, 167.05, 150.76, 129.98, 128.51, 126.84, 52.08, 41.93, 36.24, 21.70. HRMS (ESI-TOF) Calcd for C₁₂H₁₃O₄ [M−H]⁻: 221.0819. found: 221.0816.

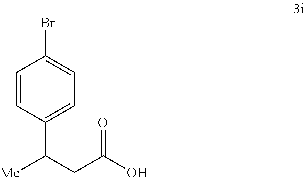 [SMILES: CC(CC(=O)O)c1ccc(Br)cc1]

### 3-(4-bromophenyl)butanoic Acid (3i)

Substrate 3i was arylated following the general arylated procedure (eluent: hexane/ethyl acetate=5/1). The product was obtained as a white solid (10.6 mg, 44% yield). ¹H NMR (400 MHz, Chloroform-d) δ 7.45 (d, J=8.4 Hz, 2H), 7.13 (d, J=8.2 Hz, 2H), 3.27 (m, 1H), 2.70-2.56 (m, 2H), 1.32 (d, J=7.0 Hz, 3H). ¹³C NMR (151 MHz, CDCl₃) δ 176.07, 144.36, 131.66, 128.54, 120.24, 41.99, 35.72, 21.84. HRMS (ESI-TOF) Calcd for C₁₀H₁₀O₂Br [M−H]⁻: 240.9869. found: 240.9862.

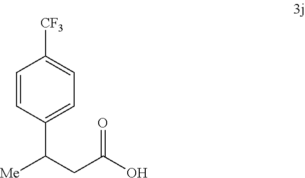 [SMILES: CC(CC(=O)O)c1ccc(C(F)(F)F)cc1]

### 3-(4-(trifluoromethyl)phenyl)butanoic Acid (3j)

Substrate 4j was arylated following the general arylated procedure (eluent: hexane/ethyl acetate=5/1). The product was obtained as a white solid (10.9 mg, 47% yield). ¹H NMR (400 MHz, Chloroform-d) δ 7.59 (d, 1=8.1 Hz, 2H), 7.37 (d, J=8.1 Hz, 2H), 3.37 (m, 1H), 2.76-2.60 (m, 2H), 1.36 (d, J=7.0 Hz, 3H). ¹³C NMR (151 MHz, CDCl₃) δ 175.70, 149.39, 128.91 (q, J=31.7 Hz), 127.15, 125.57 (q, J=4.5 Hz), 124.20 (q, J=271.8 Hz), 41.74, 36.06, 21.79. ¹⁹F NMR (376 MHz, CDCl₃) δ −65.09. HRMS (ESI-TOF) Calcd for C₁₁H₁₀O₂F₃[M−H]⁻: 231.0638. found: 231.0641.

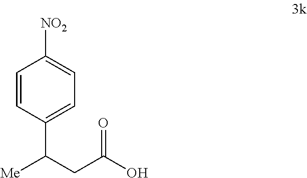 [SMILES: CC(CC(=O)O)c1ccc([N+](=O)[O-])cc1]

### 3-(4-nitrophenyl)butanoic Acid (3k)

Substrate 3k was arylated following the general arylated procedure (eluent: hexane/ethyl acetate=3/1). The product was obtained as a white solid (8.4 mg, 40% yield). ¹H NMR (600 MHz, Chloroform-d) δ 8.20 (d, J=8.7 Hz, 2H), 7.42 (d, J=8.7 Hz, 2H), 3.43 (m, 1H), 2.75-2.65 (m, 2H), 1.38 (d, J=7.0 Hz, 3H). ¹³C NMR (151 MHz, CDCl₃) δ 175.03, 152.91, 146.78, 127.73, 123.93, 41.46, 36.14, 21.72. HRMS (ESI-TOF) Calcd for C₁₀H₁₀O₄N [M−H]⁻: 208.0615. found: 208.0610.

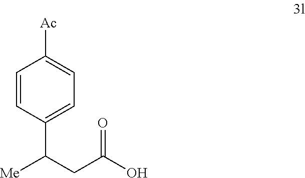 [SMILES: CC(=O)c1ccc(C(C)CC(=O)O)cc1]

### 3-(4-acetylphenyl)butanoic Acid (31)

Substrate 31 was arylated following the general arylated procedure (eluent: hexane/ethyl acetate=3/1). The product was obtained as a white solid (10.5 mg, 51% yield). ¹H NMR (600 MHz, Chloroform-d) δ 7.93 (d, J=8.3 Hz, 2H), 7.35 (d, J=8.2 Hz, 2H), 3.37 (m, 1H), 2.71 (dd, J=15.8, 7.3 Hz, 1H), 2.65 (dd, J=15.6, 8.0 Hz, 1H), 2.61 (s, 3H), 1.36 (d, J=7.0 Hz, 3H). ¹³C NMR (151 MHz, CDCl₃) δ 197.82, 176.58, 150.98, 135.69, 128.80, 127.03, 41.84, 36.20, 26.59, 21.74. HRMS (ESI-TOF) Calcd for C₁₂H₁₃O₃ [M−H]⁻: 205.0870. found: 205.0864.

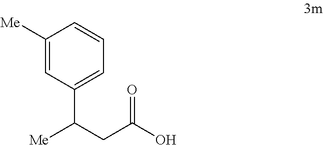 [SMILES: Cc1cccc(C(C)CC(=O)O)c1]

### 3-(m-tolyl)butanoic Acid (3m)

Substrate 3m was arylated following the general arylated procedure (eluent: hexane/ethyl acetate=5/1). The product was obtained as a white solid (11.0 mg, 62% yield). ¹H NMR (600 MHz, Chloroform-d) δ 7.23-7.20 (m, 1H), 7.08-7.01 (m, 3H), 3.29-3.23 (m, 1H), 2.70 (dd, J=15.5, 6.7 Hz, 1H), 2.60 (dd, J=15.5, 8.3 Hz, 1H), 2.36 (s, 3H), 1.34 (d, J=7.0 Hz, 3H). ¹³C NMR (151 MHz, CDCl₃) δ 176.49, 145.44, 138.14, 128.47, 127.53, 127.28, 123.68, 42.22, 36.14, 21.89, 21.48. HRMS (ESI-TOF) Calcd for C₁₁H₁₃O₂[M−H]⁻: 177.0921. found: 177.0918.

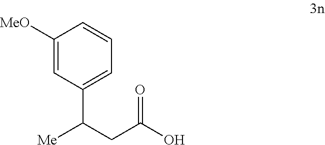 [SMILES: COc1cccc(C(C)CC(=O)O)c1]

### 3-(3-methoxyphenyl)butanoic Acid (3n)

Substrate 3n was arylated following the general arylated procedure (eluent: hexane/ethyl acetate=3/1). The product was obtained as a white solid (11.6 mg, 60% yield). ¹H NMR (400 MHz, Chloroform-d) δ 7.25 (t, J=7.8 Hz, 1H), 6.85 (d, J=7.7 Hz, 1H), 6.81-6.75 (m, 2H), 3.83 (s, 3H), 3.27 (m, 1H), 2.70 (dd, J=15.5, 6.7 Hz, 1H), 2.59 (dd, J=15.5, 8.3 Hz, 1H), 1.34 (d, J=7.0 Hz, 3H). ¹³C NMR (151 MHz, CDCl₃) δ 177.56, 159.72, 147.18, 129.57, 119.06, 112.77, 111.58, 55.18, 42.38, 36.23, 21.79. HRMS (ESI-TOF) Calcd for C₁₁H₁₃O₃[M−H]⁻: 193.0870. found: 193.0870.

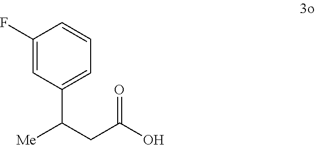 [SMILES: CC(CC(=O)O)c1cccc(F)c1]

### 3-(3-fluorophenyl)butanoic Acid (3o)

Substrate 3o was arylated following the general arylated procedure (eluent: hexane/ethyl acetate=5/1). The product was obtained as a white solid (9.8 mg, 54% yield). ¹H NMR (400 MHz, Chloroform-d) δ 7.27 (d, J=8.4 Hz, 1H), 7.03 (d, J=7.7 Hz, 1H), 6.99-6.89 (m, 2H), 3.30 (m, 1H), 2.64 (m, 2H), 1.34 (d, J=7.0 Hz, 3H). ¹³C NMR (151 MHz, CDCl₃) δ 176.22, 163.0 (d, J=246.1 Hz), 148.03 (d, J=6.0 Hz), 130.03 (d, J=7.6 Hz), 122.42 (d, J=3.0 Hz), 113.67 (d, J=21.1 Hz), 113.42 (d, J=21.1 Hz), 42.01, 35.97, 21.75. ¹⁹F NMR (376 MHz, CDCl₃) δ −115.77. HRMS (ESI-TOF) Calcd for C₁₀H₁₀O₂F [M−H]⁻: 181.0670. found: 181.0666.

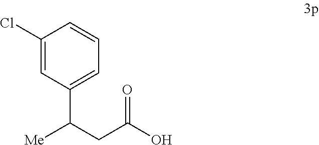 [SMILES: CC(CC(=O)O)c1cccc(Cl)c1]

### 3-(3-chlorophenyl)butanoic Acid (3p)

Substrate 3p was arylated following the general arylated procedure (eluent: hexane/ethyl acetate=5/1). The product was obtained as a white solid (10.9 mg, 55% yield). ¹H NMR (400 MHz, Chloroform-d) δ 7.24 (t, J=7.2 Hz, 3H), 7.13 (d, J=7.5 Hz, 1H), 3.28 (m, 1H), 2.65 (qd, J=15.7, 7.5 Hz, 2H), 1.34 (d, J=7.0 Hz, 3H). ¹³C NMR (151 MHz, CDCl₃) δ 176.32, 147.48, 134.34, 129.87, 126.99, 126.75, 125.01, 41.99, 35.97, 21.75. HRMS (ESI-TOF) Calcd for C₁₀H₁₀O₂Cl [M−H]⁻: 197.0375. found: 197.0367.

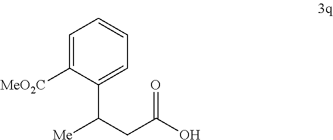 [SMILES: COC(=O)c1ccccc1C(C)CC(=O)O]

### 3-(2-(methoxycarbonyl)phenyl)butanoic Acid (3q)

Substrate 3q was arylated following the general arylated procedure (eluent: hexane/ethyl acetate=3/1). The product was obtained as a white solid (11.1 mg, 50% yield). ¹H NMR (500 MHz, Chloroform-d) δ 7.96-7.89 (m, 2H), 7.45 (d, J=7.7 Hz, 1H), 7.39 (t, J=7.6 Hz, 1H), 3.93 (s, 3H), 3.35 (m, 1H), 2.74-2.59 (m, 2H), 1.36 (d, J=6.9 Hz, 3H). ¹³C NMR (151 MHz, CDCl₃) δ 175.87, 167.18, 145.90, 131.58, 130.41, 128.64, 127.86, 127.82, 52.15, 42.17, 36.11, 21.80. HRMS (ESI-TOF) Calcd for C₁₂H₁₅O₄[M+H]⁺: 223.0965. found: 223.0960.

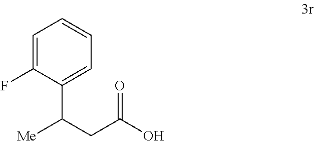 [SMILES: CC(CC(=O)O)c1ccccc1F]

### 3-(2-fluorophenyl)butanoic Acid (3r)

Substrate 3r was arylated following the general arylated procedure (eluent: hexane/ethyl acetate=5/1). The product was obtained as a white solid (9.6 mg, 53% yield). ¹H NMR (600 MHz, Chloroform-d) δ 7.26-7.19 (m, 2H), 7.11 (m, 1H), 7.04 (m, 1H), 3.58 (m, 1H), 2.77 (dd, J=15.8, 6.7 Hz, 1H), 2.66 (dd, J=15.8, 8.3 Hz, 1H), 1.37 (d, J=7.0 Hz, 3H). ¹³C NMR (151 MHz, CDCl₃) δ 176.08, 160.7 (d, J=246.1 Hz), 131.96 (d, J=15.1 Hz), 128.05 (d, J=4.5 Hz), 127.99 (d, J=9.1 Hz), 124.20 (d, J=4.5 Hz), 115.6 (d, J=22.7 Hz), 40.50, 30.21, 20.42. ¹⁹F NMR (376 MHz, CDCl₃) δ −120.76. HRMS (ESI-TOF) Calcd for C₁₀H₁₀O₂F [M−H]⁻: 181.0670. found: 181.0666.

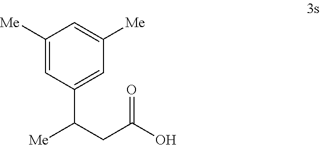 [SMILES: Cc1cc(C)cc(C(C)CC(=O)O)c1]

### 3-(3,5-dimethylphenyl)butanoic Acid (3s)

Substrate 3s was arylated following the general arylated procedure (eluent: hexane/ethyl acetate=5/1). The product was obtained as a white solid (10.7 mg, 56% yield). ¹H NMR (400 MHz, Chloroform-d) δ 6.87 (d, J=6.7 Hz, 3H), 3.23 (m, 1H), 2.69 (dd, J=15.5, 6.5 Hz, 1H), 2.58 (dd, J=15.5, 8.5 Hz, 1H), 2.32 (s, 6H), 1.32 (d, J=7.0 Hz, 3H). ¹³C NMR (151 MHz, CDCl₃) δ 177.63, 145.48, 138.03, 128.18, 124.51, 42.39, 36.03, 21.90, 21.35. HRMS (ESI-TOF) Calcd for C₁₂H₁₅O₂[M−H]⁻: 191.1077. found: 191.1074.

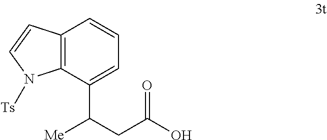 [SMILES: Cc1ccc(S(=O)(=O)n2ccc3cccc(C(C)CC(=O)O)c32)cc1]

### 3-(1-tosyl-1H-indol-7-yl)butanoic Acid (3t)

Substrate 3t was arylated following the general arylated procedure (eluent: hexane/ethyl acetate=2/1). The product was obtained as a white solid (18.6 mg, 52% yield). ¹H NMR (600 MHz, Chloroform-d) δ 7.92 (d, J=8.6 Hz, 1H), 7.79 (d, J=8.4 Hz, 2H), 7.55 (d, J=3.6 Hz, 1H), 7.38 (d, J=1.8 Hz, 1H), 7.26-7.21 (m, 2H), 7.19 (dd, J=8.6, 1.8 Hz, 1H), 6.62 (dd, J=3.7, 0.8 Hz, 1H), 3.35 (m, 1H), 2.69 (dd, J=15.6, 6.8 Hz, 1H), 2.61 (dd, J=15.6, 8.1 Hz, 1H), 2.36 (s, 3H), 1.34 (d, J=6.9 Hz, 3H). ¹³C NMR (151 MHz, CDCl₃) δ 177.45, 144.90, 140.63, 135.40, 133.60, 130.96, 129.90, 126.85, 126.57, 123.68, 119.08, 113.55, 108.90, 42.74, 36.06, 22.18, 21.58. HRMS (ESI-TOF) Calcd for C₁₉H₂₀O₄NS [M+H]⁺: 358.1108. found: 358.1115.

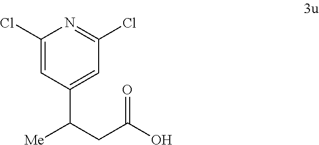 [SMILES: CC(CC(=O)O)c1cc(Cl)nc(Cl)c1]

### 3-(2,6-dichloropyridin-4-yl)butanoic Acid (3u)

Substrate 3u was arylated following the general arylated procedure (eluent: hexane/ethyl acetate=3/1). The product was obtained as a white solid (8.6 mg, 37% yield). ¹H NMR (600 MHz, Chloroform-d) δ 7.16 (s, 2H), 3.28 (q, J=7.2 Hz, 1H), 2.73-2.63 (m, 2H), 1.35 (d, J=7.0 Hz, 3H). ¹³C NMR (151 MHz, CDCl₃) δ 175.77, 160.22, 150.84, 121.50, 40.80, 35.29, 21.08. HRMS (ESI-TOF) Calcd for C₉H₁₀O₂NCl₂ [M+H]⁺: 234.0083. found: 234.0083.

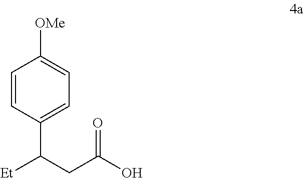 [SMILES: CCC(CC(=O)O)c1ccc(OC)cc1]

### 3-(4-methoxyphenyl)pentanoic Acid (4a)

Substrate 4a was arylated following the general arylated procedure (eluent: hexane/ethyl acetate=3/1). The product was obtained as a white solid (12.5 mg, 60% yield). ¹H NMR (600 MHz, Chloroform-d) δ 7.12 (d, J=8.7 Hz, 2H), 6.86 (d, J=8.7 Hz, 2H), 3.81 (s, 3H), 3.00-2.93 (m, 1H), 2.67 (dd, J=15.5, 6.9 Hz, 1H), 2.59 (dd, J=15.5, 8.1 Hz, 1H), 1.77-1.69 (m, 1H), 1.65-1.54 (m, 1H), 0.81 (t, J=7.4 Hz, 3H). ¹³C NMR (151 MHz, CDCl₃) δ 177.49, 158.16, 135.64, 128.39, 113.82, 55.20, 42.79, 41.16, 29.26, 11.88. HRMS (ESI-TOF) Calcd for C₁₂H₁₅O₃ [M−H]⁻: 207.1026. found: 207.1025.

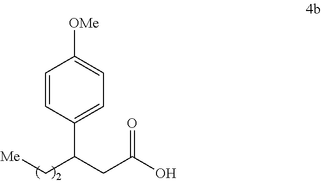

### 3-(4-methoxyphenyl)hexanoic Acid (4b)

Substrate 4b was arylated following the general arylated procedure (eluent: hexane/ethyl acetate=3/1). The product was obtained as a white solid (14.0 mg, 63% yield). ¹H NMR (600 MHz, Chloroform-d) δ 7.12 (d, J=8.7 Hz, 2H), 6.85 (d, J=8.6 Hz, 2H), 3.81 (s, 3H), 3.09-3.03 (m, 1H), 2.65 (dd, J=15.5, 7.0 Hz, 1H), 2.59 (dd, J=15.4, 8.1 Hz, 1H), 1.68-1.52 (m, 2H), 1.18 (dddd, J=12.9, 11.9, 8.4, 6.4 Hz, 2H), 0.87 (t, J=7.3 Hz, 3H). ¹³C NMR (151 MHz, CDCl₃) δ 177.74, 158.13, 135.90, 128.33, 113.82, 55.20, 41.58, 40.84, 38.57, 20.43, 13.92. HRMS (ESI-TOF) Calcd for C₁₃H₁₇O₃ [M−H]⁻: 221.1183. found: 221.1184.

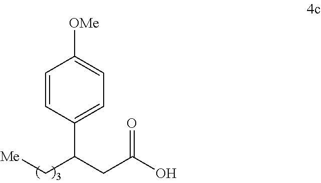

### 3-(4-methoxyphenyl)heptanoic Acid (4c)

Substrate 4c was arylated following the general arylated procedure (eluent: hexane/ethyl acetate=3/1). The product was obtained as a white solid (14.6 mg, 62% yield). ¹H NMR (400 MHz, Chloroform-d) 7.12 (d, J=8.6 Hz, 2H), 6.86 (d, J=8.6 Hz, 2H), 3.81 (s, 3H), 3.05 (q, J=7.6 Hz, 1H), 2.62 (qd, J=15.4, 7.5 Hz, 2H), 1.72-1.54 (m, 2H), 1.32-1.12 (m, 4H), 0.85 (t, J=7.2 Hz, 3H). ¹³C NMR (151 MHz, CDCl₃) δ 176.88, 157.65, 135.50, 127.86, 113.36, 54.73, 41.09, 40.62, 35.62, 29.02, 22.09, 13.49. HRMS (ESI-TOF) Calcd for C₁₄H₁₉O₃ [M−H]⁻: 235.1339. found: 235.1340.

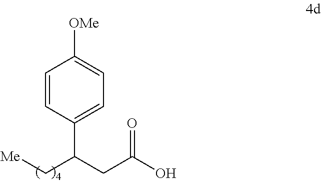

### 3-(4-methoxyphenyl)octanoic Acid (4d)

Substrate 4d was arylated following the general arylated procedure (eluent: hexane/ethyl acetate=3/1). The product was obtained as a white solid (16.3 mg, 65% yield). ¹H NMR (600 MHz, Chloroform-d) δ 7.12 (d, J=8.6 Hz, 2H), 6.85 (d, J=8.7 Hz, 2H), 3.81 (s, 3H), 3.04 (m, 1H), 2.65 (dd, J=15.4, 6.9 Hz, 1H), 2.58 (dd, J=15.4, 8.1 Hz, 1H), 1.65 (m, 1H), 1.57 (m, 1H), 1.28-1.12 (m, 6H), 0.85 (t, J=6.8 Hz, 3H). ¹³C NMR (151 MHz, CDCl₃) δ 177.46, 158.11, 135.96, 128.31, 113.82, 55.20, 41.57, 41.09, 36.33, 31.69, 26.96, 22.50, 14.03. HRMS (ESI-TOF) Calcd for C₁₅H₂₁O₃[M−H]⁻: 249.1496. found: 249.1500.

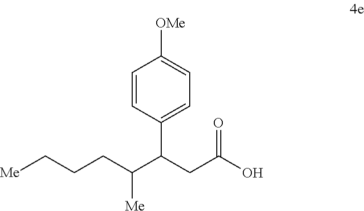 [SMILES: CCCCC(C)C(CC(=O)O)c1ccc(OC)cc1]

### 3-(4-methoxyphenyl)-4-methyloctanoic Acid (4e)

Substrate 4e was arylated following the general arylated procedure (eluent: hexane/ethyl acetate=3/1). The product was obtained as a white solid (14.7 mg, 56% yield, dr=1.0). ¹H NMR (600 MHz, Chloroform-d) δ 7.09 (t, J=8.1 Hz, 2H), 6.84 (d, J=8.8 Hz, 2H), 3.81 (d, J=0.6 Hz, 3H), 3.02 (dt, J=9.7, 6.0 Hz, 1H), 2.98 (dt, J=11.1, 5.9 Hz, 1H), 2.83-2.73 (m, 1H), 2.64 (m, 1H), 1.69 (d, J=15.8 Hz, 1H), 1.40-1.22 (m, 5H), 1.22-1.15 (m, 1H), 0.93-0.87 (m, 3H), 0.85 (t, J=7.0 Hz, 2H), 0.76 (d, J=6.8 Hz, 1H). ¹³C NMR (151 MHz, CDCl₃) δ 176.14, 176.01, 157.61, 157.55, 134.46, 133.49, 128.87, 128.54, 113.08, 112.96, 54.70, 45.82, 45.31, 37.80, 37.42, 37.32, 36.39, 33.64, 33.17, 28.83, 28.81, 22.47, 22.34, 16.23, 15.93, 13.65, 13.60. HRMS (ESI-TOF) Calcd for C₁₆H₂₃O₃[M+H]⁺: 263.1652. found: 263.1653.

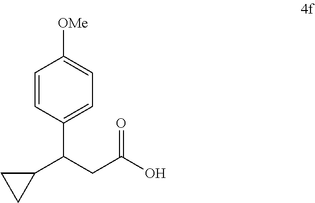 [SMILES: COc1ccc(C(CC(=O)O)C2CC2)cc1]

### 3-cyclopropyl-3-(4-methoxyphenyl)propanoic Acid (4)

Substrate 4f was arylated following the general arylated procedure (eluent: hexane/ethyl acetate=3/1). The product was obtained as a white solid (9.7 mg, 44% yield). ¹H NMR (600 MHz, Chloroform-d) δ 7.18 (d, J=8.5 Hz, 2H), 6.87 (d, J=8.6 Hz, 2H), 3.81 (s, 3H), 2.82 (dd, J=15.1, 6.8 Hz, 1H), 2.76 (dd, J=15.2, 8.3 Hz, 1H), 2.37 (q, J=8.4 Hz, 1H), 1.10-0.98 (m, 1H), 0.64-0.58 (m, 1H), 0.45 (m, 1H), 0.30 (dd, J=9.6, 4.9 Hz, 1H), 0.17 (m, 1H). ¹³C NMR (151 MHz, CDCl₃) δ 185.15, 157.76, 135.37, 127.75, 113.35, 54.75, 45.61, 40.57, 16.98, 4.80, 3.60. HRMS (ESI-TOF) Calcd for C₁₃H₁₅O₃[M−H]⁻: 219.1026. found: 219.1019.

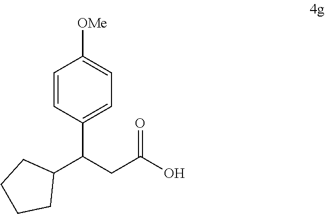 [SMILES: COc1ccc(C(CC(=O)O)C2CCCC2)cc1]

### 3-cyclopentyl-3-(4-methoxyphenyl)propanoic Acid (4g)

Substrate 4g was arylated following the general arylated procedure (eluent: hexane/ethyl acetate=3/1). The product was obtained as a white solid (11.7 mg, 47% yield). ¹H NMR (400 MHz, Chloroform-d) δ 7.12 (d, J=8.1 Hz, 2H), 6.83 (d, J=8.2 Hz, 2H), 3.80 (s, 3H), 2.89-2.76 (m, 2H), 2.59 (dd, J=15.4, 10.5 Hz, 1H), 2.01 (m, 1H), 1.95-1.82 (m, 1H), 1.73-1.50 (m, 4H), 1.41 (dd, J=19.6, 11.2 Hz, 1H), 1.28-1.20 (m, 1H), 1.10-0.98 (m, 1H). ¹³C NMR (151 MHz, CDCl₃) δ 175.56, 157.58, 135.41, 128.22, 113.15, 76.34, 54.69, 46.54, 45.86, 39.70, 30.92, 30.81, 24.81, 24.43. HRMS (ESI-TOF) Calcd for C₁₅H₁₉O₃ [M−H]⁻: 247.1339. found: 247.1338.

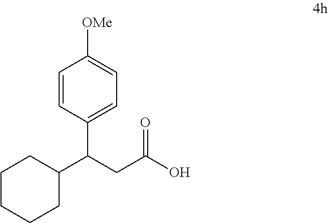 [SMILES: COc1ccc(C(CC(=O)O)C2CCCCC2)cc1]

### 3-cyclohexyl-3-(4-methoxyphenyl)propanoic Acid (4h)

Substrate 4h was arylated following the general arylated procedure (eluent: hexane/ethyl acetate=3/1). The product was obtained as a white solid (10.5 mg, 40% yield). ¹H NMR (400 MHz, Chloroform-d) δ 7.07 (d, J=8.6 Hz, 2H), 6.83 (d, J=8.6 Hz, 2H), 3.80 (s, 3H), 2.91-2.76 (m, 2H), 2.64-2.54 (m, 1H), 1.78 (dd, J=24.2, 13.3 Hz, 2H), 1.70-1.57 (m, 2H), 1.53-1.40 (m, 2H), 1.22 (dd, J=14.4, 11.1 Hz, 1H), 1.10 (q, J=12.4 Hz, 2H), 1.01-0.87 (m, 1H), 0.80 (q, J=12.2 Hz, 1H). ¹³C NMR (126 MHz, CDCl₃) δ 177.62, 158.04, 134.73, 129.11, 113.50, 55.16, 46.95, 42.98, 37.94, 31.02, 30.54, 26.44, 26.36. HRMS (ESI-TOF) Calcd for C₁₆H₂₁O₃[M−H]⁻: 261.1496. found: 261.1491.

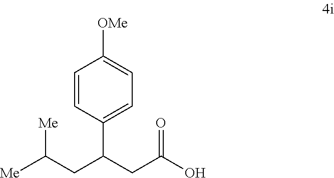 [SMILES: COc1ccc(C(CC(=O)O)CC(C)C)cc1]

### 3-(4-methoxyphenyl)-5-methylhexanoic Acid (4i)

Substrate 4i was arylated following the general arylated procedure (eluent: hexane/ethyl acetate=3/1). The product was obtained as a white solid (13.2 mg, 56% yield). ¹H NMR (600 MHz, Chloroform-d) δ 7.13 (d, J=8.3 Hz, 2H), 6.85 (d, J=8.6 Hz, 2H), 3.81 (s, 3H), 3.19-3.11 (m, 1H), 2.58 (m, 2H), 1.64-1.54 (m, 1H), 1.43 (m, 1H), 1.38-1.30 (m, 1H), 0.89 (d, J=6.4 Hz, 3H), 0.85 (d, J=6.5 Hz, 3H). ¹³C NMR (151 MHz, CDCl₃) δ 177.93, 158.12, 135.84, 128.34, 113.86, 55.19, 45.51, 42.18, 38.87, 25.26, 23.44, 21.55. HRMS (ESI-TOF) Calcd for C₁₄H₁₉O₃ [M−H]⁻: 235.1339. found: 235.1338.

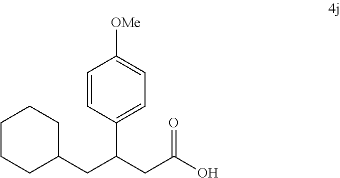 [SMILES: COc1ccc(C(CC(=O)O)CC2CCCCC2)cc1]

### 4-cyclohexyl-3-(4-methoxyphenyl)butanoic Acid (4j)

Substrate 4j was arylated following the general arylated procedure (eluent: hexane/ethyl acetate=3/1). The product was obtained as a white solid (16.0 mg, 58% yield). ¹H NMR (400 MHz, Chloroform-d) δ 7.12 (d, J=8.2 Hz, 2H), 6.85 (d, J=8.1 Hz, 2H), 3.82 (s, 3H), 3.19 (m, 1H), 2.69-2.51 (m, 2H), 1.86-1.78 (m, 1H), 1.72-1.45 (m, 7H), 1.14-1.01 (m, 3H), 0.92 (m, 2H). ¹³C NMR (126 MHz, CDCl₃) δ 177.61, 158.07, 136.05, 128.31, 113.85, 55.18, 44.12, 42.11, 38.05, 34.66, 34.00, 32.51, 26.59, 26.19, 26.06. HRMS (ESI-TOF) Calcd for C₁₇H₂₃O₃ [M−H]⁻: 275.1652. found: 275.1650.

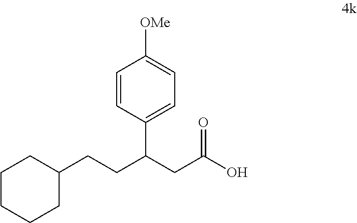 [SMILES: COc1ccc(C(CCC2CCCCC2)CC(=O)O)cc1]

### 5-cyclohexyl-3-(4-methoxyphenyl)pentanoic Acid (4k)

Substrate 4k was arylated following the general arylated procedure (eluent: hexane/ethyl acetate=3/1). The product was obtained as a white solid (17.1 mg, 59% yield). ¹H NMR (600 MHz, Chloroform-d) δ 7.11 (d, J=8.3 Hz, 2H), 6.85 (d, J=8.3 Hz, 2H), 3.81 (s, 3H), 3.00 (m, 1H), 2.65 (dd, J=15.4, 6.9 Hz, 1H), 2.58 (dd, J=15.4, 8.1 Hz, 1H), 1.70-1.58 (m, 7H), 1.15 (m, 5H), 1.01 (m, 1H), 0.82 (m, 2H). ¹³C NMR (151 MHz, CDCl₃) δ 177.05, 158.10, 136.01, 128.31, 113.82, 55.19, 41.56, 41.41, 37.62, 35.03, 33.64, 33.48, 33.09, 26.66, 26.39, 26.34. HRMS (ESI-TOF) Calcd for C₁₈H₂₅O₃ [M−H]⁻: 289.1809. found: 289.1804.

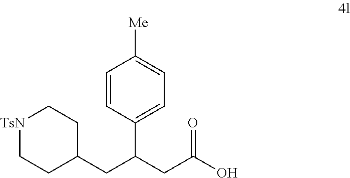 [SMILES: Cc1ccc(C(CC(=O)O)CC2CCN(S(=O)(=O)c3ccc(C)cc3)CC2)cc1]

### 3-(p-tolyl)-4-(1-tosylpiperidin-4-yl)butanoic Acid (4l)

Substrate 4l was arylated following the general arylated procedure (eluent: hexane/ethyl acetate=3/1). The product was obtained as a white solid (21.6 mg, 52% yield). ¹H NMR (600 MHz, Chloroform-d) δ 7.61 (d, J=8.1 Hz, 2H), 7.30 (d, J=8.2 Hz, 2H), 7.08 (d, J=7.7 Hz, 2H), 7.03 (d, J=7.8 Hz, 2H), 3.71 (dd, J=32.1, 11.7 Hz, 2H), 3.18-3.07 (m, 1H), 2.55 (d, J=7.4 Hz, 2H), 2.43 (s, 3H), 2.31 (s, 3H), 2.07 (m, 2H), 1.84 (d, J=13.2 Hz, 1H), 1.63 (m, 1H), 1.56-1.46 (m, 2H), 1.36-1.23 (m, 3H), 0.99-0.90 (m, 1H). ¹³C NMR (151 MHz, CDCl₃) δ 176.51, 142.88, 139.50, 135.82, 132.61, 129.07, 128.87, 127.25, 126.70, 45.79, 45.77, 41.88, 41.63, 37.87, 31.87, 31.77, 30.13, 21.03, 20.57. HRMS (ESI-TOF) Calcd for C₂₃H₃₀NO₄S [M+H]⁺: 416.1890. found: 416.1873.

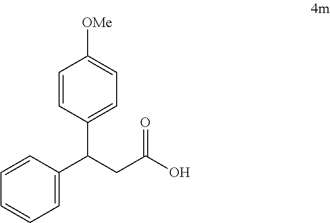 [SMILES: COc1ccc(C(CC(=O)O)c2ccccc2)cc1]

### 3-(4-methoxyphenyl)-3-phenylpropanoic Acid (4m)

Substrate 4m was arylated following the general arylated procedure (eluent: hexane/ethyl acetate=3/1). The product was obtained as a white solid (11.3 mg, 44% yield). ¹H NMR (600 MHz, Chloroform-d) δ 7.32-7.28 (m, 2H), 7.25-7.19 (m, 3H), 7.18-7.15 (m, 2H), 6.84 (d, J=8.7 Hz, 2H), 4.50 (t, J=7.9 Hz, 1H), 3.79 (s, 3H), 3.08 (d, J=7.9 Hz, 2H). ¹³C NMR (151 MHz, CDCl₃) δ 176.37, 158.23, 143.64, 135.40, 128.61, 128.60, 127.52, 126.55, 114.00, 55.22, 45.91, 40.46. HRMS (ESI-TOF) Calcd for C₁₆H₁₅O₃[M−H]⁻: 255.1026. found: 255.1024.

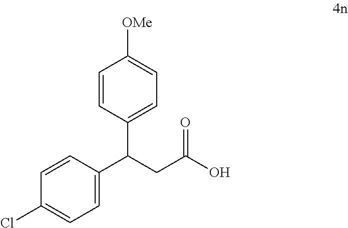 [SMILES: COc1ccc(C(CC(=O)O)c2ccc(Cl)cc2)cc1]

### 3-(4-chlorophenyl)-3-(4-methoxyphenyl)propanoic Acid (4n)

Substrate 4n was arylated following the general arylated procedure (eluent: hexane/ethyl acetate=3/1). The product was obtained as a white solid (12.5 mg, 43% yield). ¹H NMR (600 MHz, Chloroform-d) 7.27 (d, J=8.4 Hz, 2H), 7.17 (d, J=8.4 Hz, 2H), 7.14 (d, J=8.6 Hz, 2H), 6.85 (d, J=8.6 Hz, 2H), 4.48 (t, J=7.9 Hz, 1H), 3.79 (s, 3H), 3.05 (d, J=8.0 Hz, 2H). ¹³C NMR (151 MHz, CDCl₃) δ 174.57, 157.93, 141.66, 134.39, 131.90, 128.46, 128.28, 128.05, 113.65, 54.78, 44.88, 39.69. HRMS (ESI-TOF) Calcd for C₁₆H₁₄ClO₃ [M−H]⁻: 289.0637. found: 289.0628.

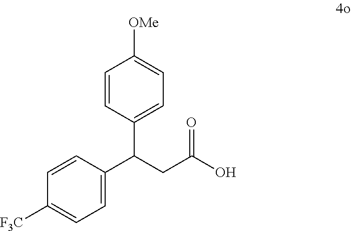 [SMILES: COc1ccc(C(CC(=O)O)c2ccc(C(F)(F)F)cc2)cc1]

### 3-(4-methoxyphenyl)-3-(4-(trifluoromethyl)phenyl)propanoic Acid (4o)

Substrate 4o was arylated following the general arylated procedure (eluent: hexane/ethyl acetate=3/1). The product was obtained as a white solid (13.0 mg, 40% yield). ¹H NMR (600 MHz, Chloroform-d) δ 7.54 (d, J=8.1 Hz, 2H), 7.34 (d, J=8.0 Hz, 2H), 7.14 (d, J=8.6 Hz, 2H), 6.85 (d, J=8.7 Hz, 2H), 4.55 (t, J=7.9 Hz, 1H), 3.78 (s, 3H), 3.08 (d, J=7.9 Hz, 2H). ¹³C NMR (151 MHz, CDCl₃) δ 175.40, 158.04, 147.21, 133.92, 128.4 (q, J=31.7 Hz), 128.10, 127.44, 125.12 (q, J=3.0 Hz), 123.66 (q, J=271.8 Hz), 113.73, 54.77, 45.28, 39.69. ¹⁹F NMR (376 MHz, CDCl₃) δ −65.13. HRMS (ESI-TOF) Calcd for C₁₇H₁₄F₃O₃ [M−H]⁻: 323.0900. found: 323.0894.

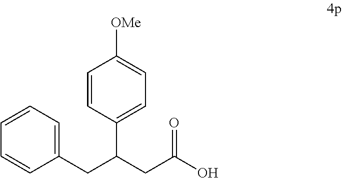 [SMILES: COc1ccc(C(CC(=O)O)Cc2ccccc2)cc1]

### 3-(4-methoxyphenyl)-4-phenylbutanoic Acid (4p)

Substrate 4p was arylated following the general arylated procedure (eluent: hexane/ethyl acetate=3/1). The product was obtained as a white solid (14.6 mg, 54% yield). ¹H NMR (600 MHz, Chloroform-d) δ 7.25-7.22 (m, 2H), 7.20-7.15 (m, 1H), 7.07 (m, 4H), 6.82 (d, J=8.6 Hz, 2H), 3.79 (s, 3H), 3.38 (m, 1H), 2.91 (d, J=7.5 Hz, 2H), 2.70 (dd, J=15.8, 6.3 Hz, 1H), 2.63 (dd, J=15.8, 8.8 Hz, 1H). ¹³C NMR (151 MHz, CDCl₃) δ 176.08, 158.24, 139.45, 135.15, 129.24, 128.41, 128.23, 126.19, 113.80, 55.19, 43.15, 42.90, 39.65. HRMS (ESI-TOF) Calcd for C₁₇H₁₇O₃ [M−H]⁻: 269.1183. found: 269.1181.

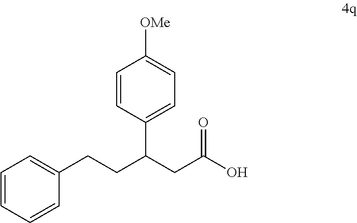 [SMILES: COc1ccc(C(CCc2ccccc2)CC(=O)O)cc1]

### 3-(4-methoxyphenyl)-5-phenylpentanoic Acid (4q)

Substrate 4q was arylated following the general arylated procedure (eluent: hexane/ethyl acetate=3/1). The product was obtained as a white solid (15.9 mg, 56% yield). ¹H NMR (600 MHz, Chloroform-d) δ 7.26 (d, J=7.5 Hz, 2H), 7.20-7.14 (m, 3H), 7.11 (d, J=6.6 Hz, 2H), 6.89 (d, J=8.6 Hz, 2H), 3.83 (s, 3H), 3.10 (m, 1H), 2.70-2.60 (m, 2H), 2.47 (m, 2H), 2.03 (m, 1H), 1.92 (m, 1H). ¹³C NMR (151 MHz, CDCl₃) δ 176.98, 158.31, 141.91, 135.26, 128.47, 128.34, 128.33, 125.81, 113.99, 55.23, 41.59, 40.76, 37.94, 33.53. HRMS (ESI-TOF) Calcd for C₁₈H₁₉O₃ [M−H]⁻: 283.1339. found: 283.1333.

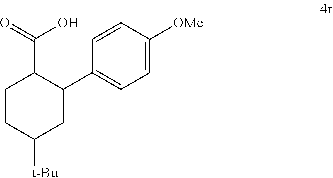 [SMILES: COc1ccc(C2CC(C(C)(C)C)CCC2C(=O)O)cc1]

### 4-(tert-butyl)-2-(4-methoxyphenyl)cyclohexane-1-carboxylic Acid (4r)

The substrate for this reaction is cis-4-(tert-butyl)cyclohexane carboxylic acid. Substrate 4r was arylated following the general arylated procedure (eluent: hexane/ethyl acetate=3/1). The product was obtained as a white solid (15.9 mg, 55% yield). ¹H NMR (600 MHz, Chloroform-d) δ 7.18 (d, J=8.6 Hz, 2H), 6.83 (d, J=8.7 Hz, 2H), 3.78 (s, 3H), 2.94 (t, J=5.3 Hz, 1H), 2.78 (dt, J=13.0, 4.0 Hz, 1H), 2.21-2.14 (m, 1H), 2.13-2.02 (m, 1H), 1.75 (m, J=17.4, 13.6, 9.2, 4.8 Hz, 2H), 1.69-1.63 (m, 1H), 1.44 (qd, J=13.2, 3.7 Hz, 1H), 1.21-1.13 (m, 1H), 0.91 (s, 9H). ¹³C NMR (151 MHz, CDCl₃) δ 177.63, 158.00, 136.50, 128.53, 113.56, 55.20, 48.42, 45.35, 44.45, 32.67, 29.84, 27.63, 27.24, 22.14.

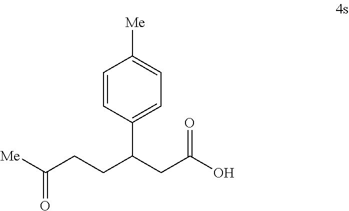 [SMILES: CC(=O)CCC(CC(=O)O)c1ccc(C)cc1]

### 6-oxo-3-(p-tolyl)heptanoic Acid (4s)

Substrate 4s was arylated following the general arylated procedure (eluent: hexane/ethyl acetate=3/1). The product was obtained as a white solid (9.8 mg, 42% yield). ¹H NMR (600 MHz, Chloroform-d) δ 7.13 (d, J=7.8 Hz, 2H), 7.07 (d, J=8.1 Hz, 2H), 3.04 (m, 1H), 2.71-2.60 (m, 2H), 2.34 (s, 3H), 2.31 (dd, J=9.9, 6.4 Hz, 1H), 2.27-2.19 (m, 1H), 2.05 (s, 3H), 2.01 (m, 1H), 1.83 (m, 1H). ¹³C NMR (151 MHz, CDCl₃) δ 208.07, 176.43, 139.11, 135.99, 128.93, 126.86, 40.99, 40.28, 29.48, 29.24, 20.58. HRMS (ESI-TOF) Calcd for C₁₄H₁₉O₃ [M+H]⁺: 235.1329. found: 235.1331.

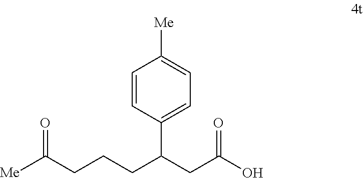 [SMILES: CC(=O)CCCC(CC(=O)O)c1ccc(C)cc1]

### 7-oxo-3-(p-tolyl)octanoic Acid (4t)

Substrate 4t was arylated following the general arylated procedure (eluent: hexane/ethyl acetate=3/1). The product was obtained as a white solid (11.7 mg, 47% yield). ¹H NMR (600 MHz, Chloroform-d) δ 7.12 (d, J=7.8 Hz, 2H), 7.08 (d, J=8.1 Hz, 2H), 3.06 (m, 1H), 2.68-2.59 (m, 2H), 2.40-2.35 (m, 2H), 2.33 (s, 3H), 2.09 (s, 3H), 1.70-1.56 (m, 2H), 1.52-1.40 (m, 2H). ¹³C NMR (151 MHz, CDCl₃) δ 208.44, 177.13, 139.75, 135.73, 128.82, 126.79, 42.92, 40.94, 40.81, 35.01, 29.36, 21.06, 20.57. HRMS (ESI-TOF) Calcd for C₁₅H₂₁O₃ [M+H]⁺: 249.1485. found: 249.1478.

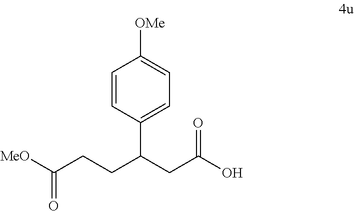 [SMILES: COC(=O)CCC(CC(=O)O)c1ccc(OC)cc1]

### 6-methoxy-6-oxo-3-(p-tolyl)hexanoic Acid (4u)

Substrate 4u was arylated following the general arylated procedure (eluent: hexane/ethyl acetate=3/1). The product was obtained as a white solid (12.0 mg, 45% yield). ¹H NMR (400 MHz, Chloroform-d) δ 7.12 (d, J=8.6 Hz, 2H), 6.86 (d, J=8.6 Hz, 2H), 3.81 (s, 3H), 3.63 (s, 3H), 3.11-3.03 (m, 1H), 2.73-2.57 (m, 2H), 2.20-2.13 (m, 2H), 2.12-1.99 (m, 1H), 1.92-1.84 (m, 1H). ¹³C NMR (151 MHz, CDCl₃) δ 173.26, 172.13, 158.00, 133.87, 127.96, 113.50, 54.77, 51.10, 40.06, 31.50, 30.75, 26.83. HRMS (ESI-TOF) Calcd for C₁₄H₁₉O₅ [M+H]⁺: 267.1227. found: 267.1222.

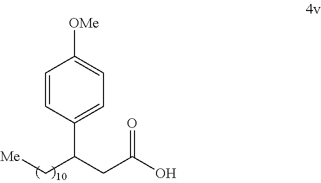

### 3-(4-methoxyphenyl)tetradecanoic Acid (4v)

Substrate 4v was arylated following the general arylated procedure (eluent: hexane/ethyl acetate=3/1). The product was obtained as a white solid (24.0 mg, 72% yield). ¹H NMR (600 MHz, Chloroform-d) δ 7.11 (d, J=8.3 Hz, 2H), 6.85 (d, J=8.5 Hz, 2H), 3.81 (s, 3H), 3.03 (m, 1H), 2.64 (dd, J=15.5, 6.9 Hz, 1H), 2.58 (dd, J=15.5, 8.1 Hz, 1H), 1.65 (m, 1H), 1.57 (m, 1H), 1.31-1.14 (m, 18H), 0.92-0.87 (m, 3H). ¹³C NMR (151 MHz, CDCl₃) δ 177.76, 158.11, 135.97, 128.32, 113.82, 55.19, 41.61, 41.08, 36.38, 31.92, 29.63, 29.62, 29.60, 29.50, 29.49, 29.35, 27.30, 22.70, 14.13. HRMS (ESI-TOF) Calcd for C₂₁H₃₃O₃[M−H]⁻: 333.2435. found: 333.2433.

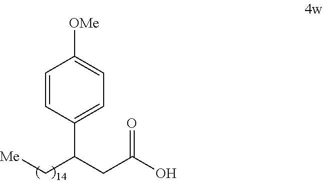

### 3-(4-methoxyphenyl)octadecanoic Acid (4w)

Substrate 4w was arylated following the general arylated procedure (eluent: hexane/ethyl acetate=3/1). The product was obtained as a white solid (26.5 mg, 68% yield). ¹H NMR (500 MHz, Chloroform-d) δ 7.14-7.09 (m, 2H), 6.88-6.83 (m, 2H), 3.81 (s, 3H), 3.04 (m, 1H), 2.65 (dd, J=15.5, 6.9 Hz, 1H), 2.58 (dd, J=15.5, 8.1 Hz, 1H), 1.62 (m, 2H), 1.31-1.22 (m, 26H), 0.90 (d, J=6.9 Hz, 3H). ¹³C NMR (126 MHz, CDCl₃) δ 178.12, 158.11, 135.98, 128.32, 113.82, 55.19, 41.68, 41.09, 36.38, 31.94, 29.70, 29.67, 29.64, 29.61, 29.51, 29.38, 27.31, 22.71, 14.13. HRMS (ESI-TOF) Calcd for C₂₅H₄₁O₃ [M−H]⁻: 389.3061. found: 389.3058.

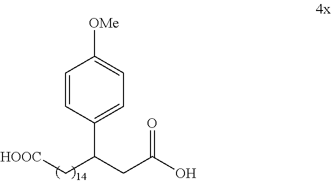

### 3-(4-methoxyphenyl)octadecanedioic Acid (4x)

Boc-decomposed after the reaction. Substrate 4x was arylated following the general arylated procedure (eluent: hexane/ethyl acetate=3/1). The product was obtained as a white solid (22.7 mg, 54% yield). ¹H NMR (500 MHz, Chloroform-d) δ 7.12 (d, J=8.7 Hz, 2H), 6.86 (d, J=8.6 Hz, 2H), 3.81 (s, 3H), 3.06 (m, 1H), 2.68-2.56 (m, 2H), 2.39-2.36 (m, 2H), 1.66 (t, J=7.4 Hz, 4H), 1.30-1.24 (m, 22H). ¹³C NMR (126 MHz, CDCl₃) δ 179.85, 178.41, 158.12, 136.08, 128.30, 113.85, 55.21, 41.83, 40.94, 35.91, 33.98, 29.38, 29.33, 29.31, 29.10, 29.08, 28.94, 28.73, 26.99, 24.60. HRMS (ESI-TOF) Calcd for C₂₅H₃₉O₅[M−H]⁻: 419.2803. found: 419.2800.

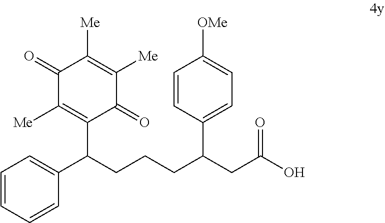 [SMILES: COc1ccc(C(CCCC(C2=C(C)C(=O)C(C)=C(C)C2=O)c2ccccc2)CC(=O)O)cc1]

### 3-(4-methoxyphenyl)-7-phenyl-7-(2,4,5-trimethyl-3,6-dioxocyclohexa-1,4-dien-1-yl)heptanoic Acid (4y)

Substrate 4y was arylated following the general arylated procedure (eluent: hexane/ethyl acetate=3/1). The product was obtained as a white solid (23.9 mg, 52% yield, dr=1.4). ¹H NMR (600 MHz, Chloroform-d) δ 7.28-7.20 (m, 4H), 7.20-7.16 (m, 1H), 7.08-7.02 (m, 2H), 6.80 (dd, J=11.0, 8.6 Hz, 2H), 4.26-4.17 (m, 1H), 3.79 (s, 1H), 3.79 (s, 2H), 3.02 (m, 1H), 2.62-2.54 (m, 2H), 2.25-2.17 (m, 1H), 2.13-2.05 (m, 2H), 2.00 (d, J=3.2 Hz, 6H), 1.95 (m, 3H), 1.78-1.68 (m, 1H), 1.67-1.56 (m, 1H), 1.17-1.06 (m, 1H). ¹³C NMR (151 MHz, CDCl₃) δ 187.42, 187.36, 186.57, 186.48, 176.97, 176.95, 157.71, 157.69, 145.29, 145.07, 141.81, 141.60, 141.11, 140.95, 140.31, 140.28, 139.69, 139.69, 135.04, 134.90, 127.82, 127.81, 127.78, 127.44, 127.30, 125.69, 125.67, 113.39, 113.36, 54.72, 54.71, 42.88, 42.67, 41.19, 41.08, 40.34, 40.23, 35.77, 35.64, 31.03, 30.87, 25.38, 25.26, 12.09, 12.05, 11.95, 11.93, 11.89, 11.89. HRMS (ESI-TOF) Calcd for C₂₉H₃₁O₅[M−H]⁻: 459.2177. found: 459.2192.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A method of β-methylene C(sp³)-H arylation of aliphatic carboxylic acids, comprising treating an aliphatic carboxylic acid with an aryl iodide in the presence of a bidentate pyridine-pyridone ligand (L) and a Pd(II) catalyst.

2. The method of claim 1, wherein the method of β-methylene C(sp³)-H arylation of aliphatic carboxylic acids occurs according to the following reaction scheme: 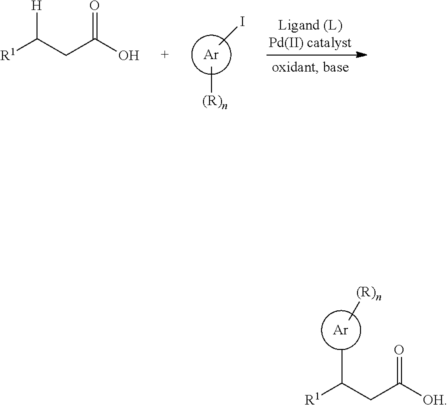
wherein:
Ar is (C₆-C₁₀)aryl;
R is H, halo, OH, (C₁-C₆)alkyl, halo (C₁-C₆)alkyl, (C₁-C₆)heteroalkyl, halo (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, Ac, —OAc, —NO₂, Ts, —OTBS, —C(═O)H, NH₂, —C(═O)(C₁-C₆)alkyl, —C(═O)O(C₁-C₆)alkyl, oxo, —S(═O)(C₁-C₆)alkyl, —S(═O)₂(C₁-C₆)alkyl, or —C(═O)NH(C₁-C₆)alkyl;
R¹ is H or selected from the group consisting of (C₁-C₁₂)alkyl, (C₂₋₁₂)alkenyl, (C₂₋₁₂)alkynyl, (C₁-C₁₀)heteroalkyl, hetero (C₂₋₁₀)alkenyl, hetero (C₂₋₁₀)alkynyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, each independently and optionally substituted with one or more R¹′;
each R¹′ is independently OH, halo, CN, (C₁-C₆)alkyl, halo (C₁-C₆)alkyl, (C₁-C₆)heteroalkyl, (C₃-C₇)cycloalkyl, (C₃-C₇)heterocycloalkyl, (C₁-C₆)alkyl (C₃-C₇)cycloalkyl, (C₁-C₆)alkyl (C₃-C₇)heterocycloalkyl, (C₆-C₁₀)aryl, (C₅-C₆)heteroaryl, (C₁-C₆)alkyl (C₆-C₁₀)aryl, (C₁-C₆)alkyl (C₅-C₆)heteroaryl, NH₂, —C(═O)(C₁-C₆)alkyl, —C(═O)O(C₁-C₆)alkyl, oxo, Ts, —S(═O)₂F, —OS(═O)₂F, or —S(═O)₂(C₁-C₆)alkyl; and
n is 0, 1, 2, or 3.

3. The method of claim 2, wherein Ar is Ph.

4. The method of either claim 2 or claim 3, wherein n is 0.

5. The method of either claim 2 or claim 3, wherein n is 1.

6. The method of claim 5, wherein R is (C₁-C₆)alkyl.

7. The method of claim 5, wherein R is Me.

8. The method of claim 5, wherein R is halo.

9. The method of claim 5, wherein R is CF₃.

10. The method of claim 5, wherein R is —OMe.

11. The method of claim 5, wherein R is Ac.

12. The method of claim 5, wherein R is Ph.

13. The method of claim 5, wherein R is —C(═O)OMe.

14. The method of claim 5, wherein R is NO₂.

15. The method of claim 5, wherein R is Ts.

16. The method of claim 5, wherein R is —OTBS.

17. The method of any one of claims 2-16, wherein R¹ is (C₁-C₁₂)alkyl optionally substituted with one or more R¹′.

18. The method of claim 17, wherein R¹ is (C₁-C₆)alkyl.

19. The method of claim 17 or claim 18, wherein R¹′ is —O(C₁-C₆)alkyl.

20. The method of claim 17 or claim 18, wherein R¹′ is —C(═O)O(C₁-C₆)alkyl.

21. The method of claim 17 or claim 18, wherein R¹′ is —C(═O)(C₁-C₆)alkyl.

22. The method of claim 17 or claim 18, wherein R¹′ is halo.

23. The method of any one of claims 2-16, wherein R¹ is (C₁-C₁₂)heteroalkyl optionally substituted with one or more R¹′.

24. The method of any one of claims 2-16, wherein R¹ is (C₆-C₁₀)aryl optionally substituted with one or more R¹′.

25. The method of claim 24, wherein R¹ is Ph optionally substituted with one or more R¹′.

26. The method of any one of claims 2-16, wherein R¹ is (C₁-C₆)alkyl (C₆-C₁₀)aryl optionally substituted with one or more R¹′.

27. The method of claim 26, wherein R¹ is Bn optionally substituted with one or more R¹′.

28. The method of any one of claims 2-16, wherein R¹ is (C₃-C₇)cycloalkyl optionally substituted with one or more R¹′.

29. The method of any one of claims 2-16, wherein R¹ is (C₁-C₆)alkyl (C₃-C₇)cycloalkyl optionally substituted with one or more R¹′.

30. The method of any one of claims 23-29, wherein R¹′ is (C₁-C₆)alkyl, —OMe, CF₃, or halo.

31. The method of claim 2, wherein the method of β-methylene C(sp³)-H arylation of aliphatic carboxylic acids occurs according to the following reaction scheme:

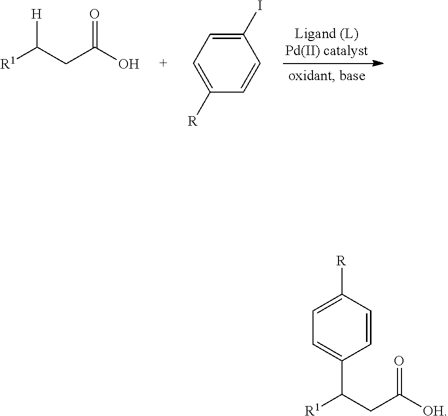

32. The method of claim 31, wherein R is (C₁-C₆)alkyl.

33. The method of claim 32, wherein R is Me.

34. The method of claim 31, wherein R is F.

35. The method of claim 31, wherein R is Cl.

36. The method of claim 31, wherein R is Br.

37. The method of claim 31, wherein R is —OMe.

38. The method of claim 31, wherein R is CF₃.

39. The method of claim 31, wherein R is Ph.

40. The method of claim 31, wherein R is NO₂.

41. The method of claim 31, wherein R is —OAc.

42. The method of claim 31, wherein R is —C(═O)OMe.

43. The method of claim 31, wherein R is —OTBS.

44. The method of any one of claims 31-43, wherein R¹ is (C₁-C₁₂)alkyl optionally substituted with one or more R¹′.

45. The method of claim 44, wherein R¹′ is —C(═O)O(C₁-C₆)alkyl.

46. The method of claim 44, wherein R¹′ is —C(═O)(C₁-C₆)alkyl.

47. The method of any one of claims 31-43, wherein R¹ is (C₁-C₁₂)heteroalkyl optionally substituted with one or more R¹′.

48. The method of any one of claims 31-43, wherein R¹ is (C₆-C₁₀)aryl optionally substituted with one or more R¹′.

49. The method of claim 48, wherein R¹ is Ph optionally substituted with one or more R¹′.

50. The method of claim 49, wherein R¹′ is (C₁-C₆)alkyl.

51. The method of claim 49, wherein R¹′ is halo.

52. The method of claim 49, wherein R¹′ is —C(═O)O(C₁-C₆)alkyl or —C(═O)(C₁-C₆)alkyl.

53. The method of claim 49, wherein R¹′ is Me, F, Cl, Br, —OMe, CF₃, —C(═O)Me, or —C(═O)Ot-Bu.

54. The method of any one of claims 31-43, wherein R¹ is (C₁-C₆)alkyl (C₆-C₁₀)aryl optionally substituted with one or more R¹′.

55. The method of claim 54, wherein R¹ is Bn optionally substituted with one or more R¹′.

56. The method of any one of claims 31-43, wherein R¹ is (C₃-C₇)cycloalkyl optionally substituted with one or more R¹′.

57. The method of any one of claims 31-43, wherein R¹ is (C₁-C₆)alkyl (C₃-C₇)cycloalkyl, optionally substituted with one or more R¹′.

58. The method of any one of claims 31-43, wherein R¹ is (C₁-C₆)alkyl (C₃-C₇)heterocycloalkyl optionally substituted with one or more R¹′.

59. The method of any one of claims 54-58, wherein R¹′ is (C₁-C₆)alkyl, halo, —O(C₁-C₆)alkyl, CF₃, —C(═O)(C₁-C₆)alkyl, or —C(═O)O(C₁-C₆)alkyl.

60. The method of any one of claims 54-59, wherein R¹′ is Me, F, Cl, Br, —OMe, CF₃, —C(═O)Me, or —C(═O)Ot-Bu.

61. The method any one of claims 2-60, wherein the Pd(II) catalyst is Pd(OAc)₂.

62. The method any one of claims 2-61, wherein the base is Na₂HPO₄.

63. The method any one of claims 2-62, wherein the solvent is HFIP.

64. The method any one of claims 2-63, wherein the oxidant is a mixture of AgOAc and Ag₂CO₃.

65. The method any one of claims 2-63, wherein the oxidant is AgOAc.

66. The method any one of claims 2-63, wherein the oxidant is Ag₂CO₃.

67. The method any one of claims 2-66, wherein the reaction temperature is approximately 80-120° C.

68. The method of claim 67, wherein the reaction temperature is approximately 100° C.

69. The method of any one of claims 1-68, wherein L is selected from the group consisting of:

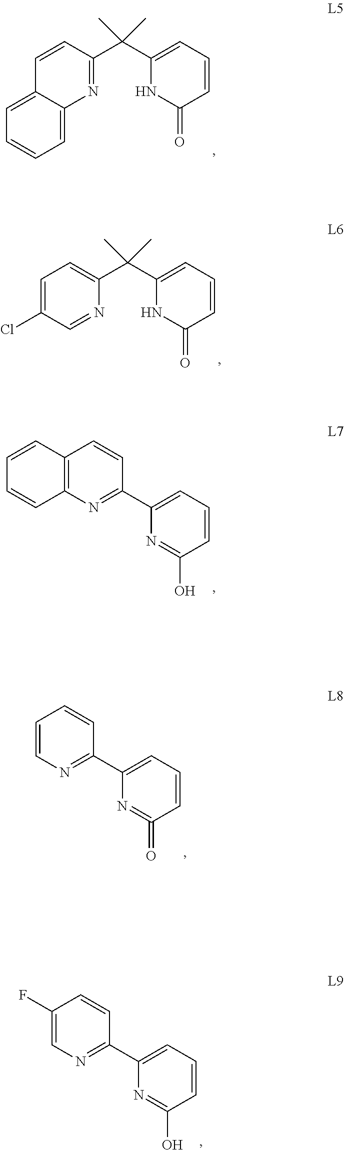

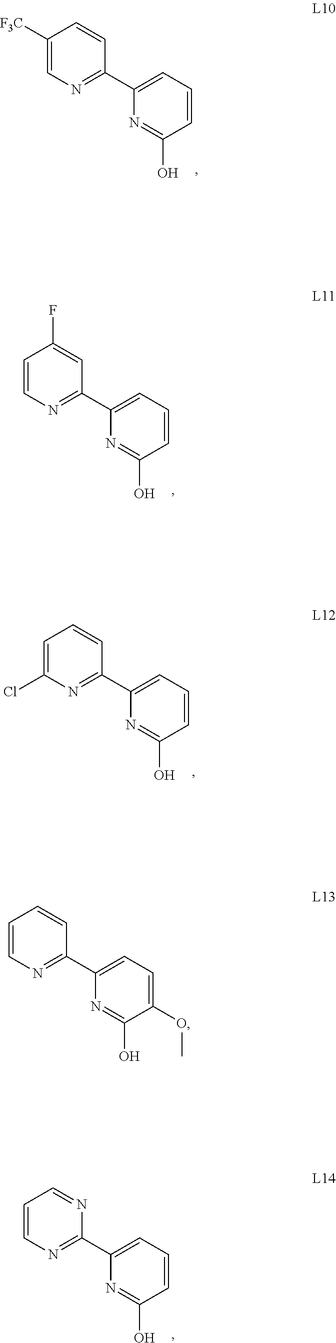 [SMILES: Oc1cccc(-c2ccc(C(F)(F)F)cn2)n1 ; Oc1cccc(-c2cc(F)ccn2)n1 ; Oc1cccc(-c2cccc(Cl)n2)n1 ; COc1ccc(-c2ccccn2)nc1O ; Oc1cccc(-c2ncccn2)n1]

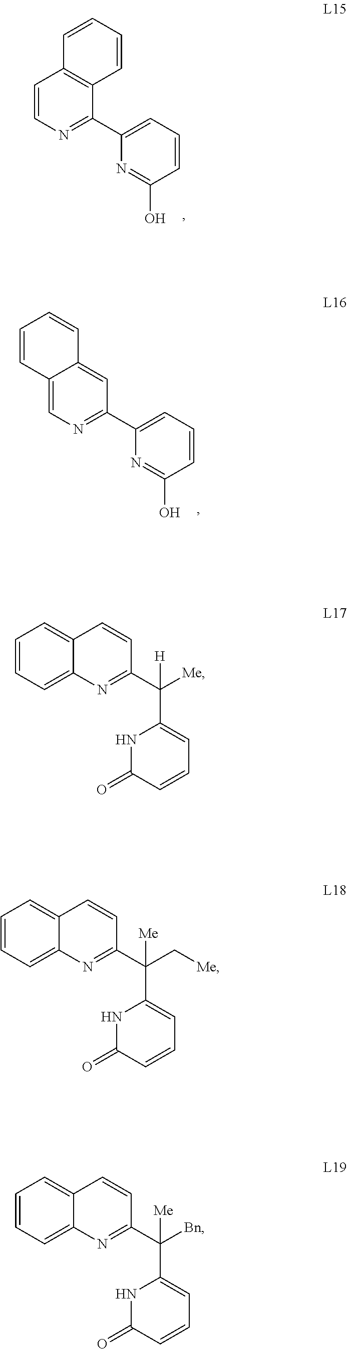 [SMILES: Oc1cccc(-c2nccc3ccccc23)n1 ; Oc1cccc(-c2cc3ccccc3cn2)n1 ; CC(c1ccc2ccccc2n1)c1cccc(=O)[nH]1 ; CCC(C)(c1ccc2ccccc2n1)c1cccc(=O)[nH]1 ; CC(Cc1ccccc1)(c1ccc2ccccc2n1)c1cccc(=O)[nH]1]

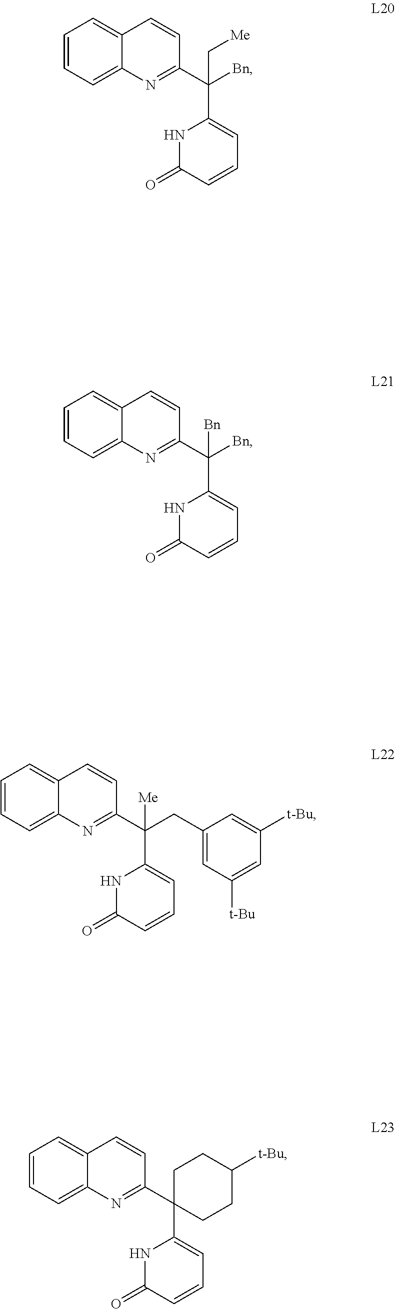 [SMILES: CCC(Cc1ccccc1)(c1ccc2ccccc2n1)c1cccc(=O)[nH]1 ; O=c1cccc(C(Cc2ccccc2)(Cc2ccccc2)c2ccc3ccccc3n2)[nH]1 ; CC(C)(C)c1cc(CC(C)(c2ccc3ccccc3n2)c2cccc(=O)[nH]2)cc(C(C)(C)C)c1 ; CC(C)(C)C1CCC(c2ccc3ccccc3n2)(c2cccc(=O)[nH]2)CC1]

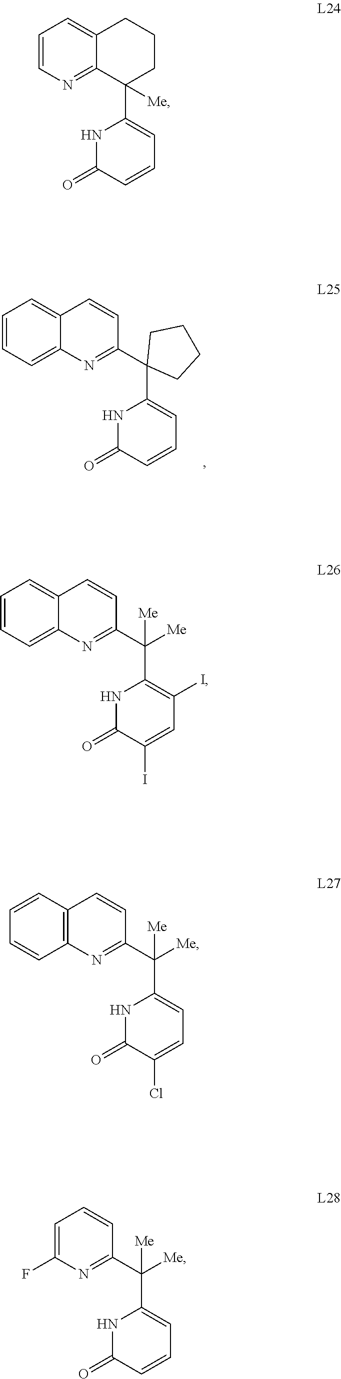 [SMILES: CC1(c2cccc(=O)[nH]2)CCCc2cccnc21 ; O=c1cccc(C2(c3ccc4ccccc4n3)CCCC2)[nH]1 ; CC(C)(c1ccc2ccccc2n1)c1[nH]c(=O)c(I)cc1I ; CC(C)(c1ccc2ccccc2n1)c1ccc(Cl)c(=O)[nH]1 ; CC(C)(c1cccc(F)n1)c1cccc(=O)[nH]1]

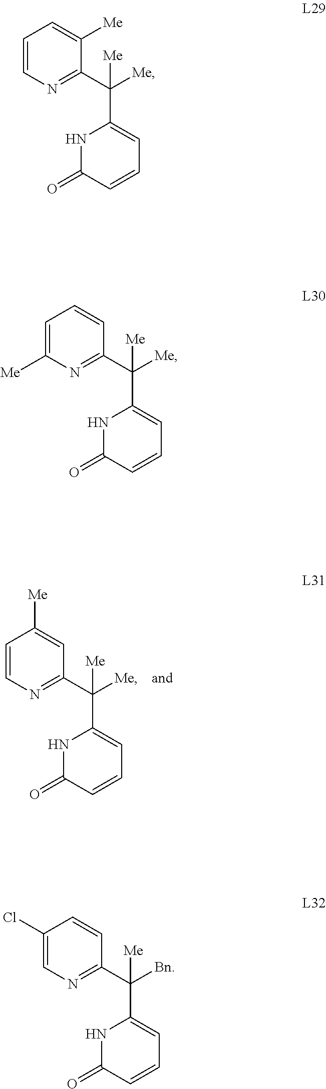 [SMILES: Cc1cccnc1C(C)(C)c1cccc(=O)[nH]1 ; Cc1cccc(C(C)(C)c2cccc(=O)[nH]2)n1 ; Cc1ccnc(C(C)(C)c2cccc(=O)[nH]2)c1 ; CC(Cc1ccccc1)(c1ccc(Cl)cn1)c1cccc(=O)[nH]1]

70. The method of claim 69, wherein L is L7.

71. The method of claim 2, wherein the method of β-methylene C(sp³)-H arylation of aliphatic carboxylic acids occurs according to the following reaction scheme:

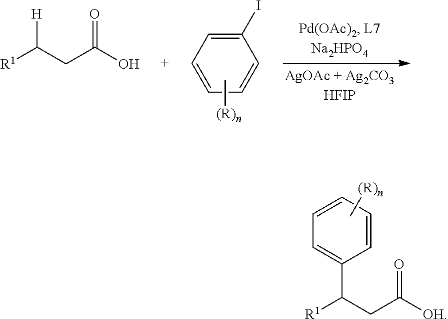

72. The method of claim 71, wherein n is 0.

73. The method of claim 71, wherein n is 1.

74. The method of claim 71, wherein n is 2.

75. The method of any one of claims 71-74, wherein R¹ is (C₁-C₁₂)alkyl optionally substituted with one or more R¹′.

76. The method of any one of claims 71-75, wherein R¹′ is Me, F, Cl, Br, —OMe, CF₃, —C(═O)Me, or —C(═O)Ot-Bu.

77. Any method of β-methylene C(sp³)-H arylation of aliphatic carboxylic acids as disclosed herein.
